(19) United States
(12) Patent Application Publication
(10) Pub. No.: US 2008/0319459 A1
(43) Pub. Date: Dec. 25, 2008

(54) Laparoscopic Instrument

(76) Inventor: Azad Al-najjar, Vasteras (SE)

Correspondence Address: YOUNG & THOMPSON, 209 Madison Street, Suite 500, ALEXANDRIA, VA, 22314, US

(21) Appl. No.: 12/158,716

(22) PCT Filed: Dec. 20, 2006
(86) PCT No.: PCT/SE2006/050606

§ 371 Date: Aug. 1, 2008

(30) Foreign Application Priority Data
Dec. 23, 2005 (SE) 0502883-2

(51) Int. Cl. A61B 17/04

(52) U.S. Cl. 606/148

(57) ABSTRACT

The present invention relates to a system of laparoscopic instruments that provides the possibility of an effective and fast laparoscopic/endoscopic suturing method in order to facilitate 5 laparoscopic/endoscopic operation. The system consists of the following main parts: a novel, specially made laparoscopic instrument that in one end has a specially made needle through which a thread can be fed; a novel, specially made laparoscopic instrument that in one end has a specially made needle that has the capability of receiving and holding 10 the end of the thread, a novel, special clips machine and a new clip; a novel thread feeder, eventually integrated in a forceps; and specially made double forceps.

### TECHNICAL AREA

The present invention relates to a suture instrument, in particular a laparoscopic suture instrument, with simplified drawing of the suture thread, as well as double and single pliers for drawing the suture, and clips instrument for fixating the suture thread. The invention further relates to methods for fixating the suture thread and method for saturation.

### TECHNICAL BACKGROUND

There are large similarities of putting together tissues and putting together two pieces of cloth by sewing. In both cases two different pieces are brought together with a thread or wire and after this the thread is knotted in order not to slip but to hold the connection together. In the case with tissues of course special tools are used. The thread and needle are interconnected to an integral unit for facilitating the lead-through through the tissue and the knotting process and are of course made of suitable material. FIG. 1 shows a sketch of needle and thread. In most cases such a pre-prepared thread and needle is used, which are interconnected and packed, but there may also be needles with an “eye” on the backside through which the thread must be guided. There are different designs for the needle adapted for different operations and positions.

The process is done as shown in FIG. 2. The needle is held with one hand with the help of a needle holder and guides the needle through the first tissue structure and then through the second tissue structure, FIG. 2.a. The needle is guided completely through the tissues and goes from one side to the other, FIG. 2.b. In this position the tissues are hanging on the thread and may be brought together by pulling the thread tight, FIG. 2.c. The ends of the thread are brought closes and are knotted together several times in order to ascertain that they will not slip, FIG. 2.d. After that the unnecessary part of the thread is cut. The needle and the remaining thread are used for the further work of applying another stitch.

As is apparent from FIG. 2 and according to above, the basic process is relatively simple. For open surgery with tissues that are close to the surface the ends of the threads may be knotted by hand. On the other hand, instruments are required for knotting thread ends if the tissue is on deeper locations, where it is more difficult to get access to knot by hand.

By the above steps, the latter is the most extensive. There are different methods for knotting the thread in a safe way. In FIG. 3 different ways of working with the knot are shown. In the two-hand method both hands are active. The method is effective and simple, but requires enough space so that the surgeon can perform it. In the one-hand method only one hand is active and performs the most important part of the knotting. The other hand is only for holding and pulling of the thread when so required.

During knotting with instruments, the most important movements and the lead-through of the thread are done with the help of the needle holder. The other hand is only responsible for holding the thread or pulling it, exactly as the passive hand during one-hand knotting. In those cases where the tissues are difficult to reach, two instruments have to be used, one needle holder and one tissue forceps or an artery forceps. This is shown to the right in FIG. 3.

During a laparoscopic operation instruments with long handles are used in order to perform all steps of the operation. After a needle, thread and other instruments have been inserted through the operation port, the needle is held with a first instrument, (laparoscopic needle holder and guides it through the tissues that are to be sewn together, FIG. 4.a. After that the needle is gripped with a second instrument laparoscopic dissecting forceps from the other side and at the same time lets go with the first instrument needle holder. The second instrument laparoscopic dissecting forceps pulls the needle up and the tissues will hang on the thread.

As a summary, the most important sewing-steps in a laparoscopic operation are the following:
1. The instrument I holds the needle. The needle and the hanging thread are on side A of the tissues.
2. With the help of instrument I the needle is guided through the tissues that are to be sewn together, FIG. 4.a.
3. Instrument II receives the needle, FIG. 4.b.
4. Instrument I releases the needle.
5. Instrument II pulls the needle through the tissues so that the needle goes through side A to side B, FIG. 4.c.
6. Instrument II releases the needle and instead grips the thread at a suitable distance from the tissue on side B, FIG. 5.a.
7. With suitable movements the thread is twisted around instrument I, FIG. 5.b.
8. Instrument I grips the thread on side A of the tissue, FIG. 5.c.
9. Instrument I pulls the end of the thread back so that a knot is created, FIG. 5.d.
10. Both instruments are pulled so that the tissues are brought together.
11. A similar process is repeated 2 or more times so that at least three knots on each other are obtained so that the risk of them loosing the grip is minimized, FIG. 5.f-5.h.
   Remark: FIGS. 3 and 5 are taken from the book “Sutur När Var Hur, The University publisher, Uppsala Sweden 1976.

To make a knot during a laparoscopic operation is one of the most time-consuming parts of the operation. Limited possibilities of movement, two-dimensional viewing via a TV screen and the long handle in the instrument are some important factors that contribute to a higher degree of complexity. It is often that a knot may take more than 10 minutes and the surgeon has to try several times before succeeding. Longer operation time is a large load for both the patient and the surgeon.

It should be mentioned that despite different machines being developed for a faster and more efficient laparoscopic suture, traditional use of needle and suture still are the general methods used and always work, although with low efficiency. In the following text, principles, methods and instruments are presented, which facilitate the sewing methods in a flexible way.

The aim of the present invention is to improve the laparoscopic sewing process and to reduce the operational time considerably.

### SUMMARY OF THE PRESENT INVENTION

The present invention relates to development of a new laparoscopic suture method and new laparoscopic suture instruments for performing this laparoscopic method. The needle in a traditional suture instrument is intended to make holes in the tissue, to penetrate, and then transfer the thread from one side to the other. This is normally done in that the needle itself has to be transferred from one side to the other. With laparoscopy this is done by at least two instruments and the procedure for tying the thread is very difficult. In this invention the problem has been solved by using two different principles and use of adopted laparoscopic instruments for facilitating the work. In order to understand the principles of the invention, first two examples of suggested instruments are presented. After this the principles of the invention are presented.

In particular the present invention relates to a laparoscopic suture instrument comprising a needle, characterised in that the instrument is arranged with a handle that receives and manoeuvres said needle and that the needle is arranged in order for a thread to pass in it, whereby the needle may be used for transferring the thread through the tissues without the needle, as a whole has to be transferred from one side to the other.

According to a preferred embodiment the needle is equipped with a flexible pipe for feeding steel wire through it from the handle.

According to a preferred embodiment the needle is exchangeably fixated to the handle.

According to a preferred embodiment the position of the needle is arranged to be changed from a rest position to an active position via said handle.

According to a preferred embodiment the tip of the needle is arranged with a removable protective device for protection during insertion through an operation port.

According to a preferred embodiment the needle is hollow.

According to a preferred embodiment the needle is arranged with a movable steel wire in the hollow space.

According to a preferred embodiment the needle is arranged with a movable suture thread in the hollow space.

According to a preferred embodiment the end of the steel wire is formed as a ring, whereby the ring widens and becomes larger when the steel wire is fed out of the needle and becomes smaller and can hold a thread when it is pulled back.

According to a preferred embodiment the device for holding the thread is a hole in the needle.

According to a preferred embodiment the needle is arranged with a hole on its side adjacent the tip of the needle, through which hole the ring-shaped end of the steel wire or a suture is arranged to be fed out.

According to a preferred embodiment the needle is arranged with a hole in its tip, through which hole the ring-shaped end of the steel wire or a suture is arranged to be fed out.

According to a preferred embodiment the tip of the ring-shaped end of the steel wire constitutes a tip for the needle when it is fed through one or more tissues.

According to a preferred embodiment, the steel wire is present through the whole instrument and can be fed in and out via a device connected to the instrument.

According to a preferred embodiment the thread feeding device is arranged to further be able to slow down the feeding.

According to a further aspect of the invention it comprises a laparoscopic double forceps for use during laparoscopic operation, characterised in that each jaw is arranged to be maneuvered separately via an externally arranged control means.

According to a preferred embodiment this consists of two movable jaws with a fixed jaw arranged between them.

According to a preferred embodiment the control means are arranged on an external handle and are arranged to control the two movable jaws separately.

According to a further aspect of the invention, it comprises a laparoscopic single forceps for laparoscopic suture work comprising a device for feeding suture thread.

According to a preferred embodiment the thread feeding is arranged to be slowed down.

According to a preferred embodiment the device has a knife for cutting the thread.

According to a preferred embodiment it has a movable jaw for holding one of the ends of the thread.

According to preferred embodiment the movement of the movable jaw of the single forceps is controlled and can be mounted on one end of the single forceps.

According to a preferred embodiment it contains a channel running substantially along the entire length of the instrument.

According to a preferred embodiment a thread is arranged to be fed from one end to the other end.

According to another aspect of the invention, it comprises a method of joining the ends of a suture thread after it has been fed through tissue on order to obtain a suture, characterised in that a metal piece/clip is squeezed around both ends of the thread and holds them together.

According to a preferred embodiment the metal piece/clip forms a ring around the ends of the threads.

According to a preferred embodiment the ends of the metal piece/ends of the clip pass each other after the squeezing to overlapping position.

According to a preferred embodiment the method is performed by a laparoscopic clip instrument.

According to yet an aspect of the invention it comprises a laparoscopic clip instrument, whereby it comprises two jaws that are bent in axial direction and overlap or cross each other in an active position when the jaws are brought together against each other.

According to a preferred embodiment it contains a device for pressing the jaws of the instrument against each other.

According to a preferred embodiment it contains a device for pressing the jaws of the instrument against each other when the jaws of the instrument are pulled backwards.

According to a preferred embodiment a device is disclosed for pushing back the jaws of the instrument to an original position/passive position.

According to a preferred embodiment the jaws are arranged to be pushed apart when they are pushed forward.

A preferred embodiment comprises a device for feeding clips into the bent jaw.

A preferred embodiment comprises a rod connected to a movable handle mounted on one end of the instrument.

A preferred embodiment comprises a device for keeping and storing clips when the instrument is used.

A preferred embodiment comprises a container/magazine for clips arranged with a spring arranged to push up the clips one after the other through an opening in the shaft of the clip instrument.

A preferred embodiment contains clips with at least two arms that are in two or more plane levels.

Finally the invention comprises a method for laparoscopic suturing, where an instrument according to the claims is used, whereby a needle is fed through a first tissue and thereafter through a second tissue, after which a thread is fed through the needle and is received by a forceps on the other side of the tissues, the needle is withdrawn from the tissues and a knotting to a suture is performed.

In a preferred embodiment the method comprises to feeding the needle through a first tissue and then through a second tissue, after which the needle receives a thread end via a device intended for this and pulls the thread through the tissues when the needle is pulled out the path that was inserted.

### BRIEF DESCRIPTION OF THE DRAWINGS

In the following detailed description, reference will be made to the accompanying drawings, of which

FIG. 1 shows a conventional suturing needle and thread,

FIG. 2 shows the principles of a surgical sewing process,

FIG. 3 shows knotting of a traditional suture,

FIG. 4 shows feeding through of a needle with the help of instruments,

FIG. 5 shows knotting of suture with laparoscopic instruments,

FIG. 6 shows a detailed view of a front end of a laparoscopic suture instrument according to the invention,

FIG. 7 shows the instrument according to FIG. 6 in a perspective view,

FIG. 8 shows a similar view as FIG. 7,

FIG. 9 shows a further view of the instrument of FIGS. 6 to 8,

FIG. 10 shows a view of the handle of the instrument according to FIGS. 6-9,

FIG. 11 shows a detailed view of the needle turning device of the instrument of FIGS. 6-9,

FIG. 12 shows a detailed view of a thread feeding device comprised in the instrument of FIGS. 6-9,

FIG. 13 shows components of the needle turning device according to FIG. 11,

FIG. 14 shows an exploded view of the instrument of FIGS. 6-9,

FIG. 15 shows a rear view of a laparoscopic double forceps according to the present invention,

FIG. 16 shows a perspective view of the instrument of FIG. 15,

FIG. 17 shows a side view of the instrument of FIGS. 15-16,

FIG. 18 shows a further view of the instrument of FIGS. 15-17,

FIG. 19 shows a detailed view of the front end of the instrument of FIGS. 15-18,

FIG. 20 shows an exploded view of the instrument of FIGS. 15-18,

FIG. 21 shows a perspective view of a laparoscopic single forceps according to the invention,

FIG. 22 shows another perspective view of the instrument of FIG. 21,

FIG. 23 shows an exploded view of the instrument of FIGS. 21-22,

FIG. 24 shows a detailed view of the front end of a laparoscopic clip instrument according to the invention, this fig and the fig later show are copies from the internet to show that the clips is standard tools in the surgery,

FIG. 25 shows the laparoscopic clip instrument in a side view,

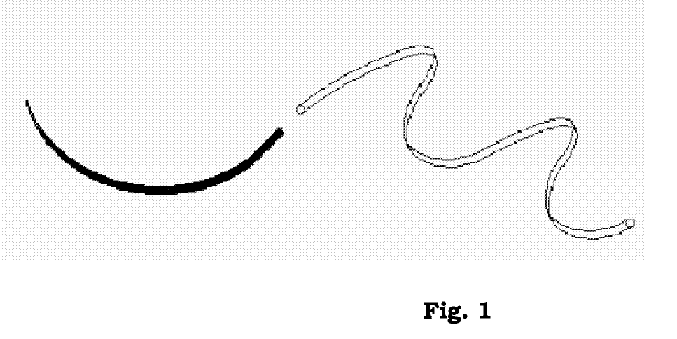

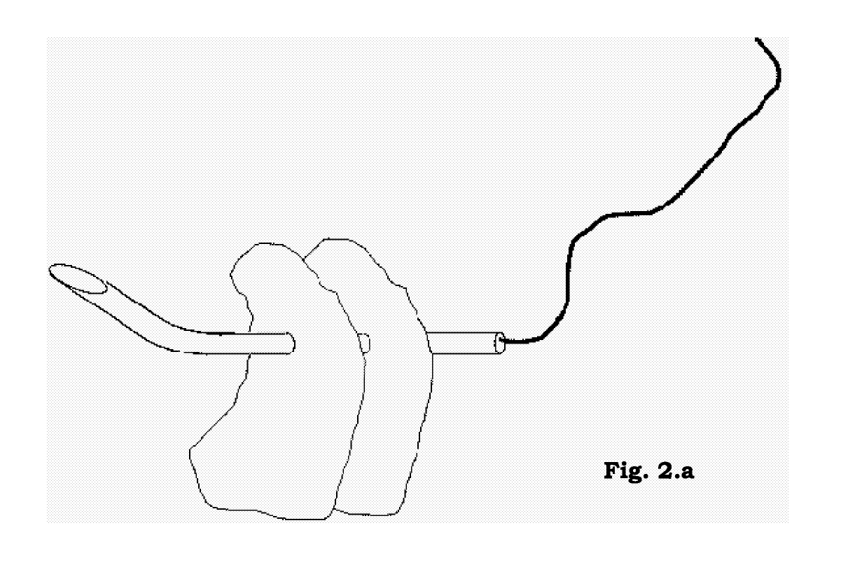

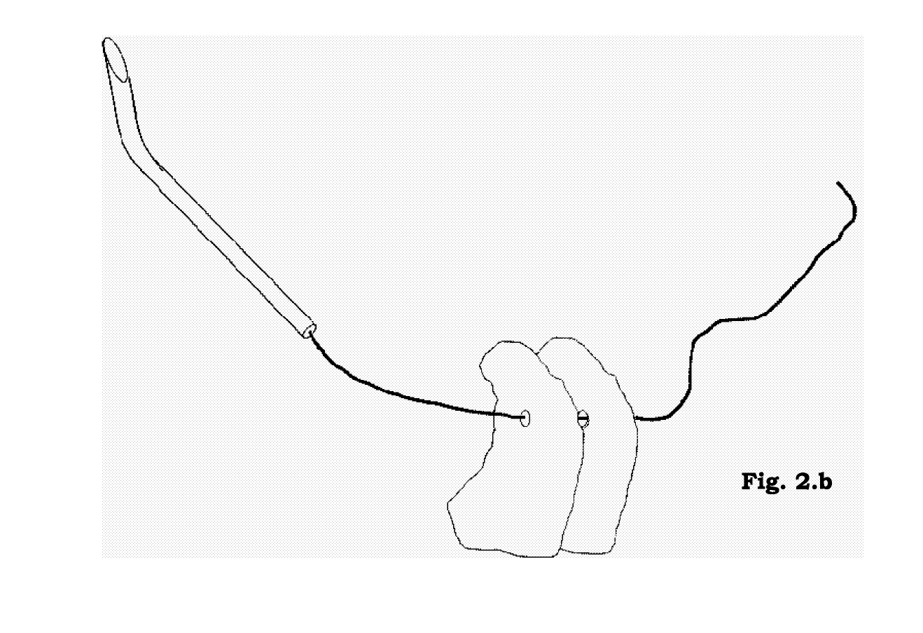

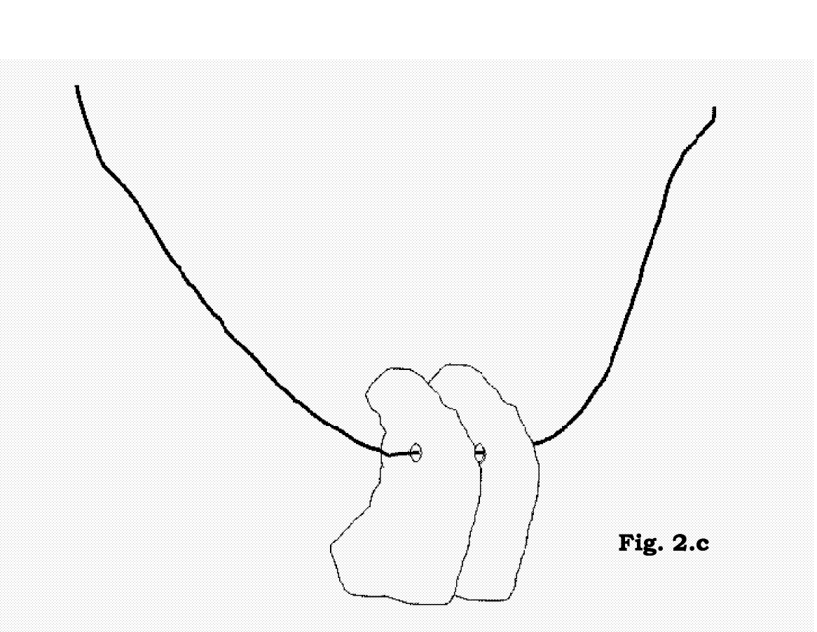

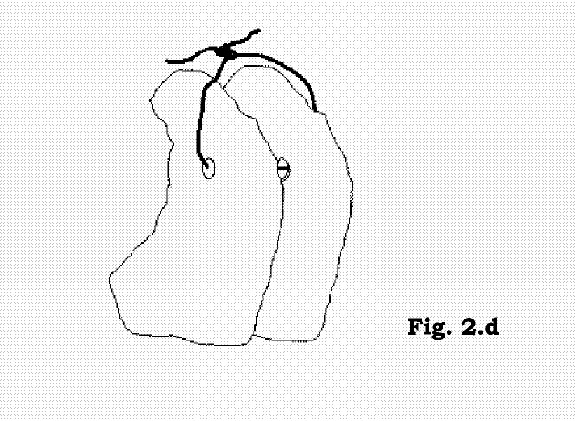

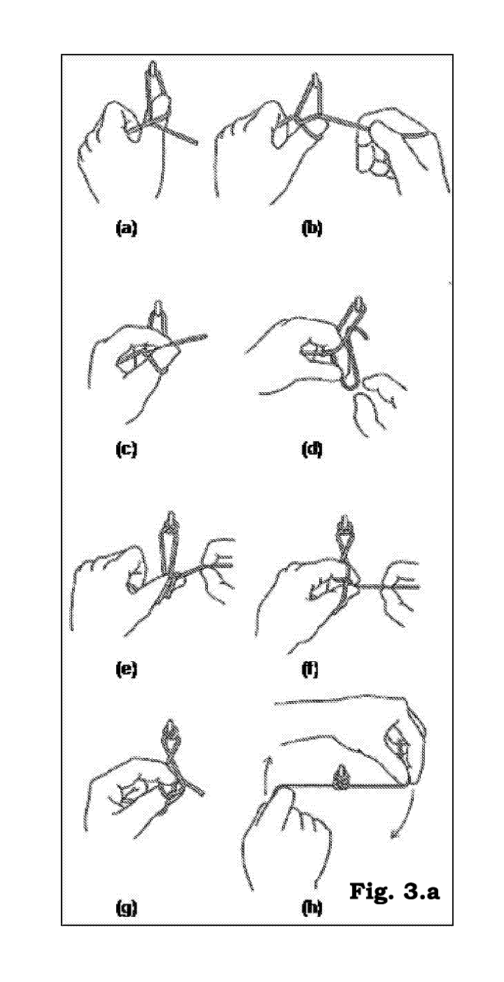

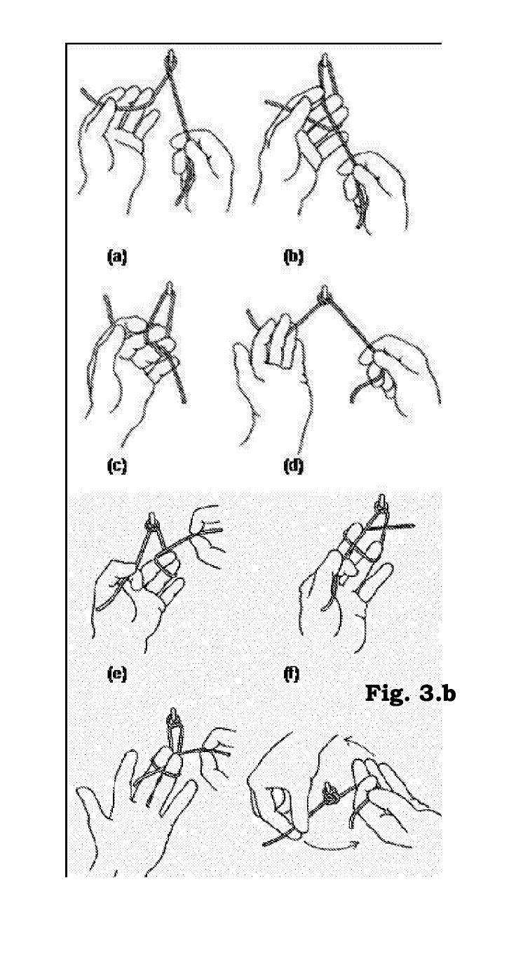

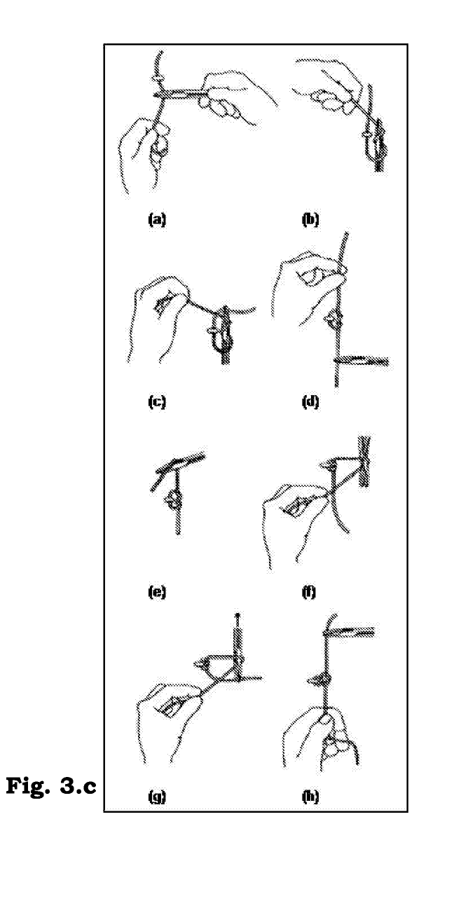

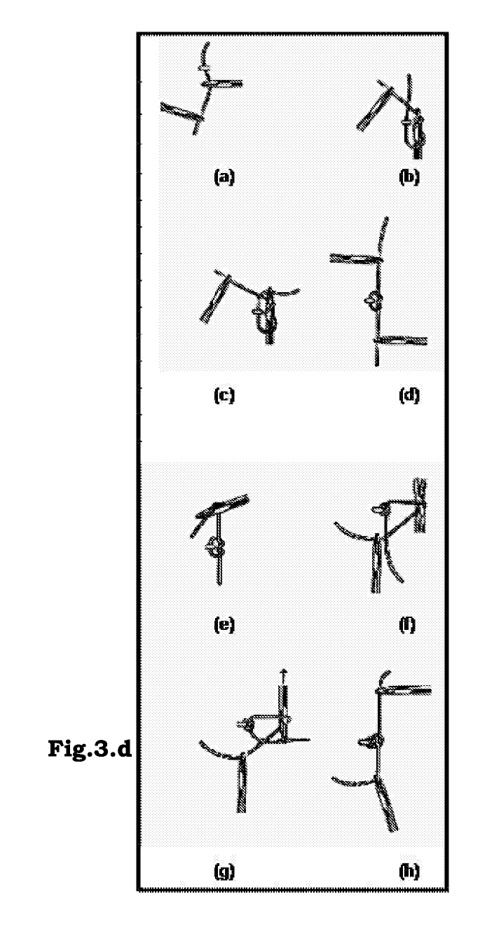

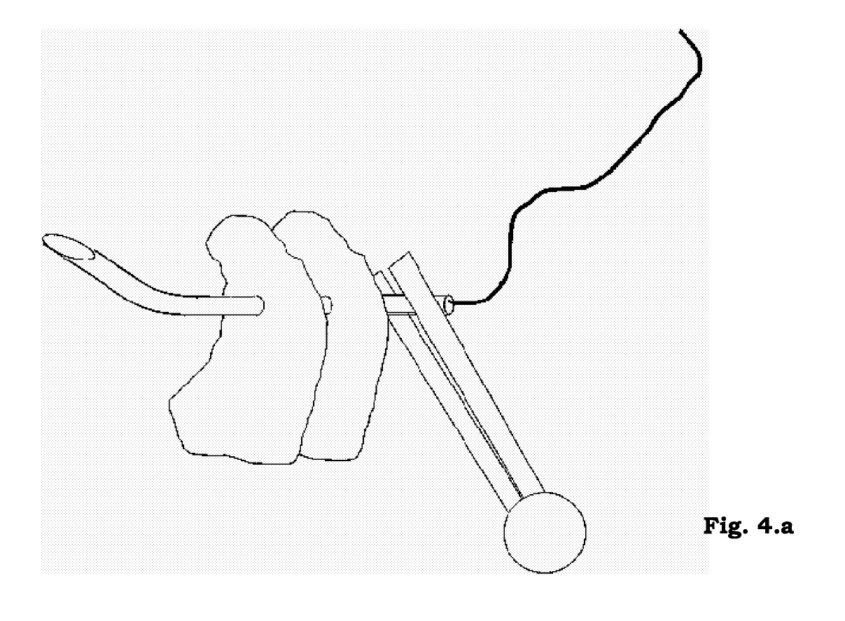

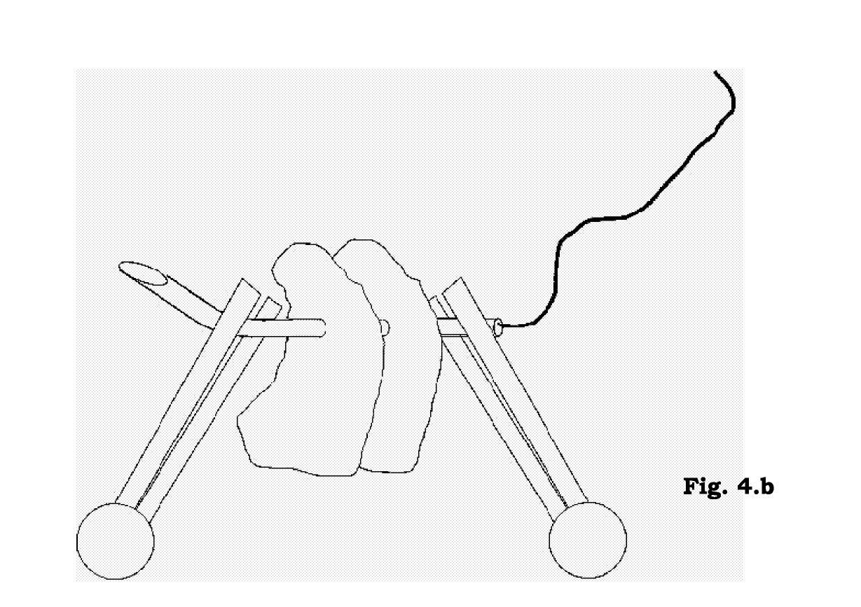

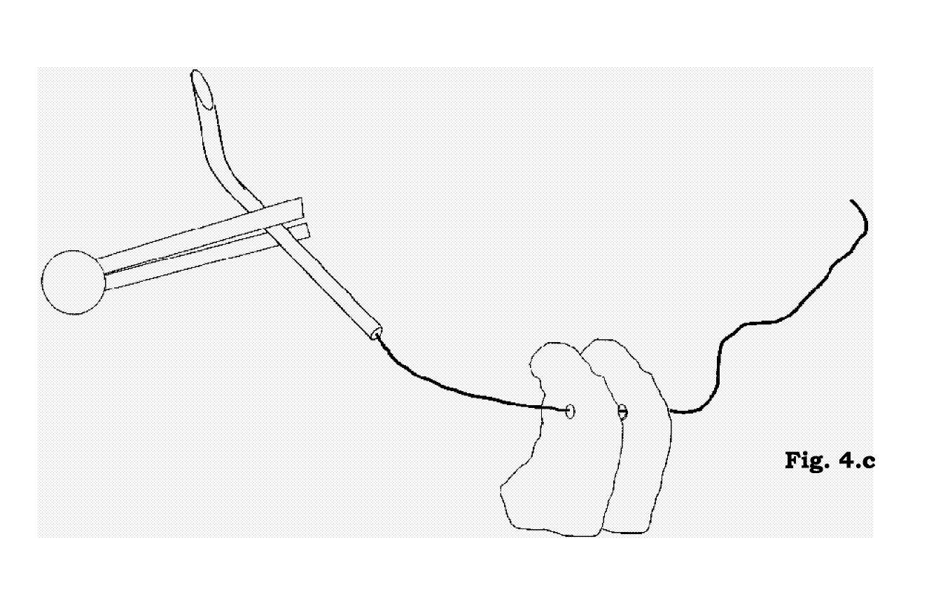

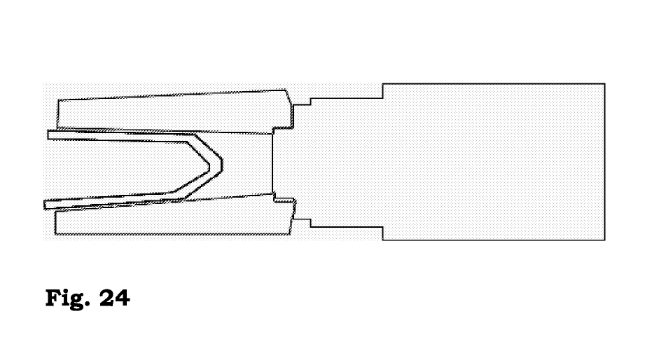

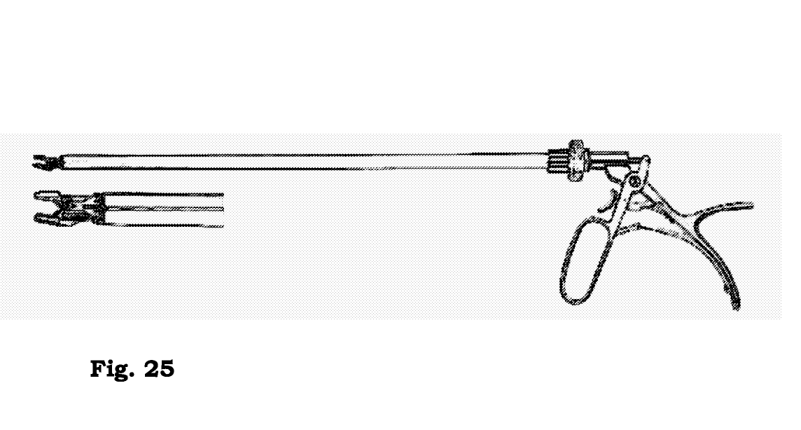

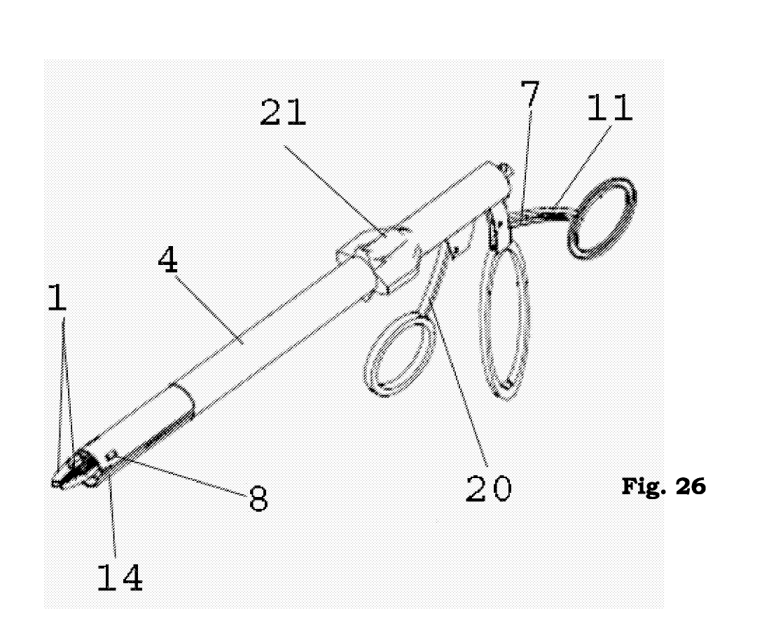

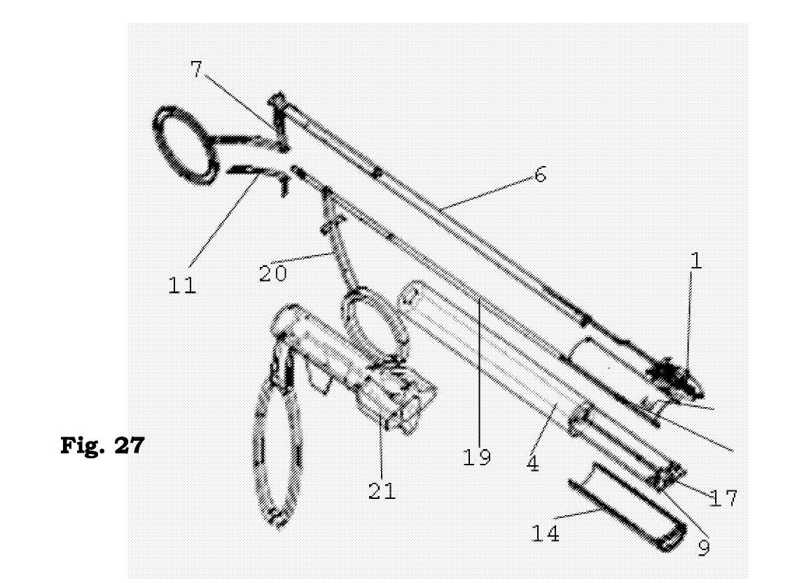

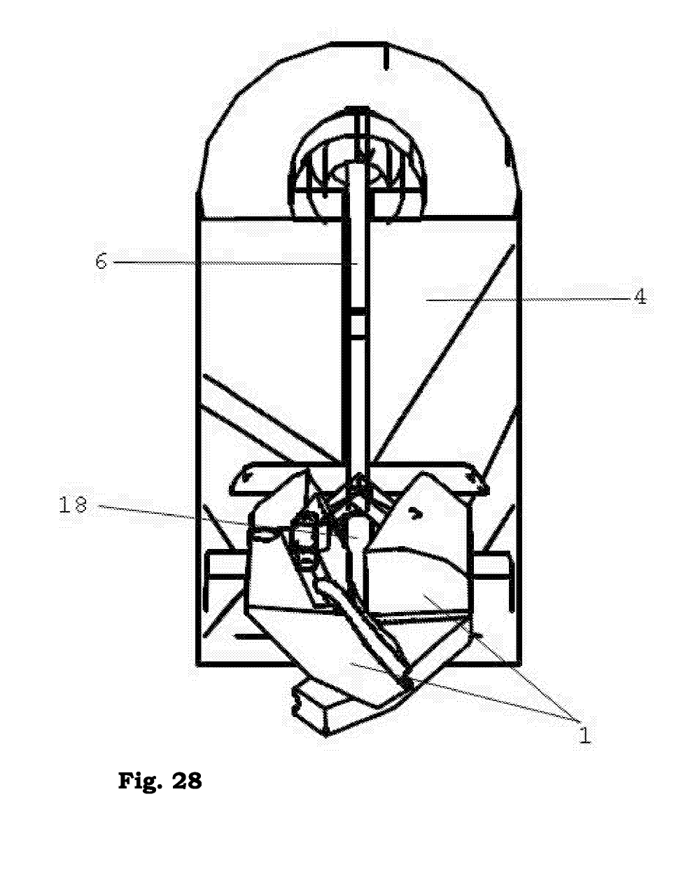

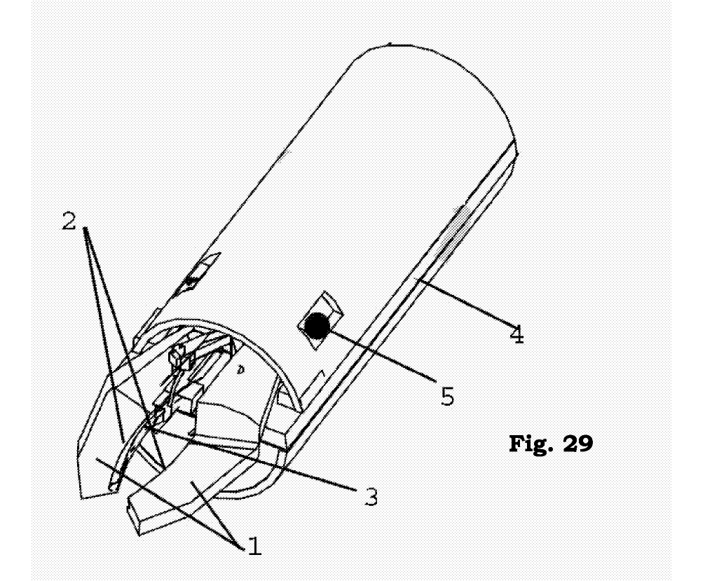

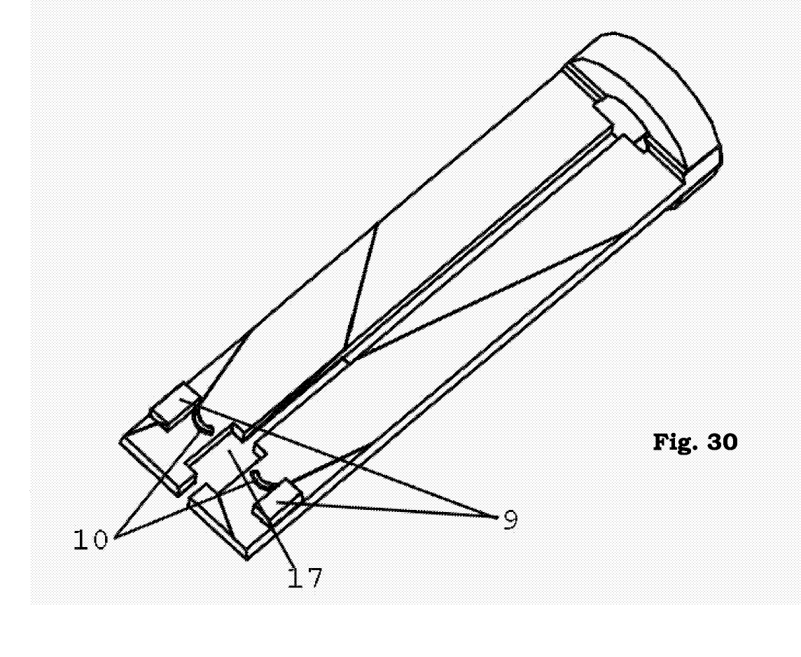

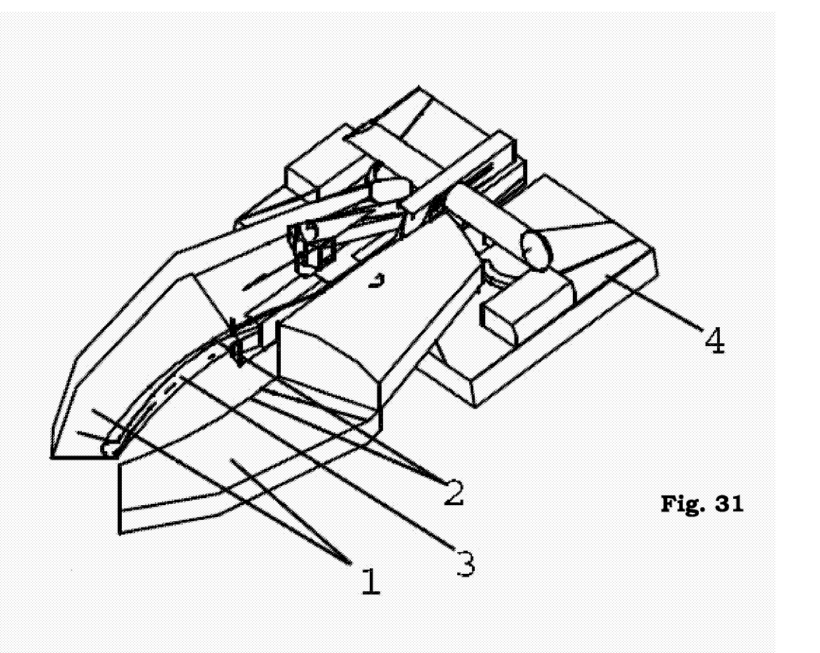

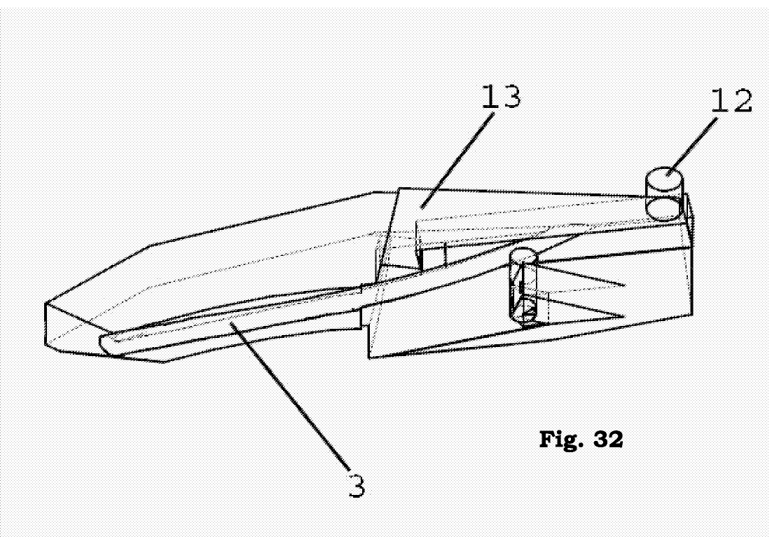

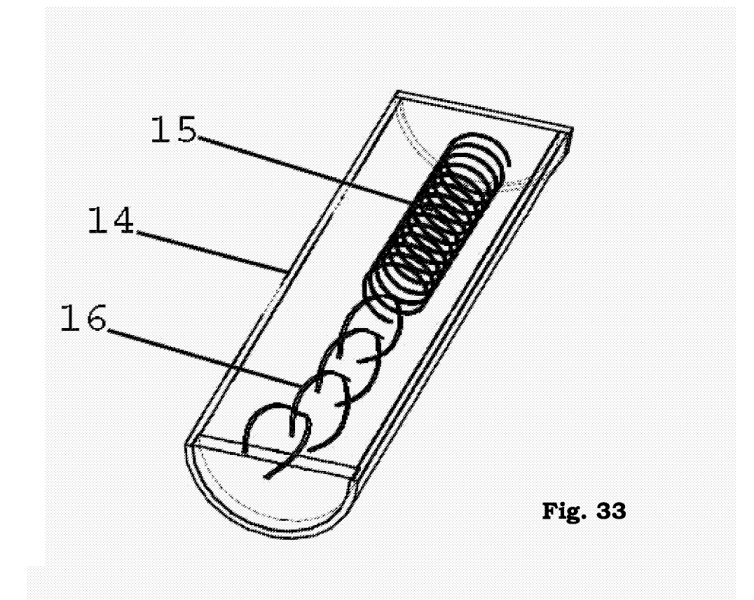

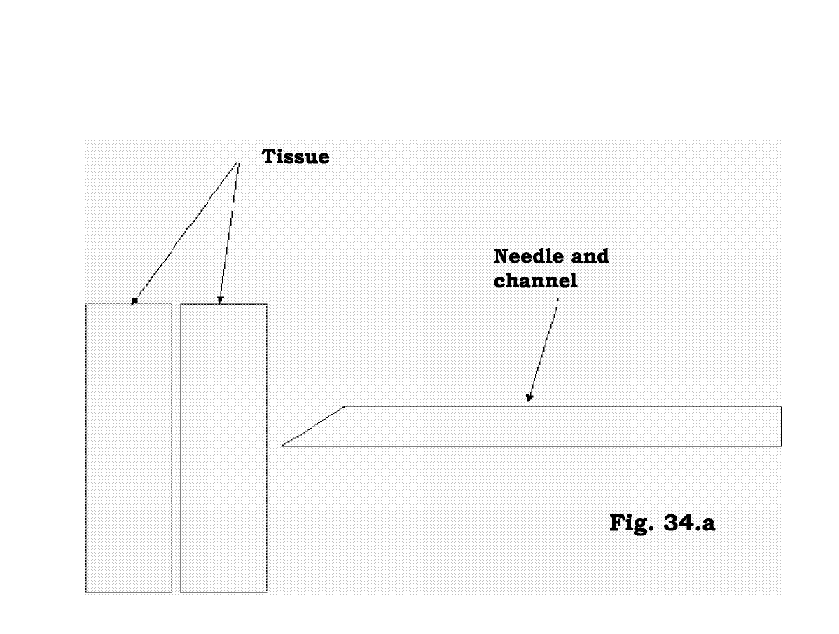

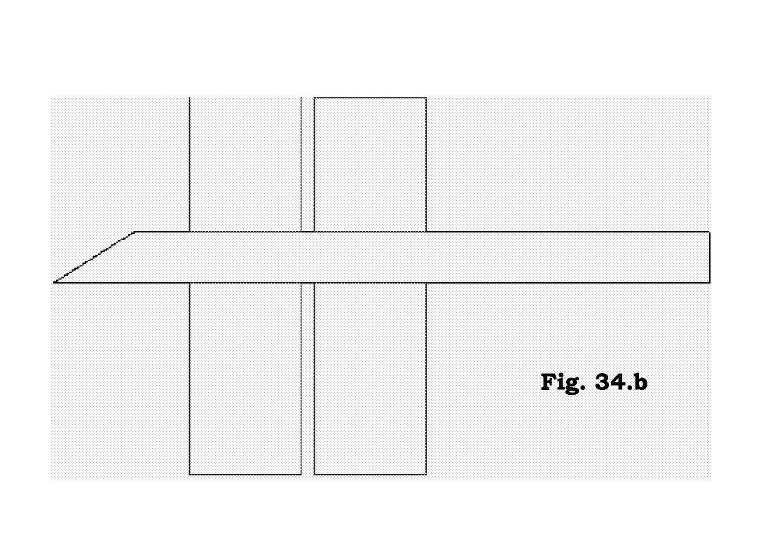

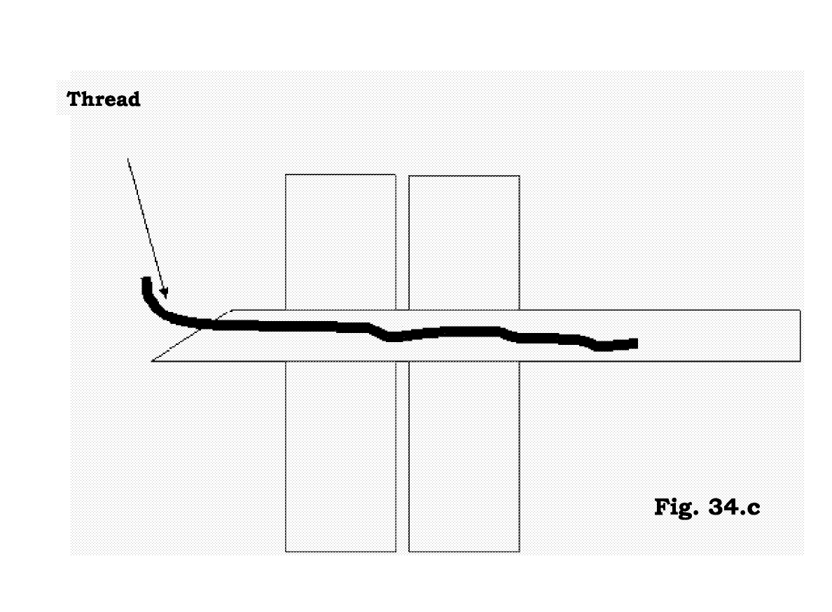

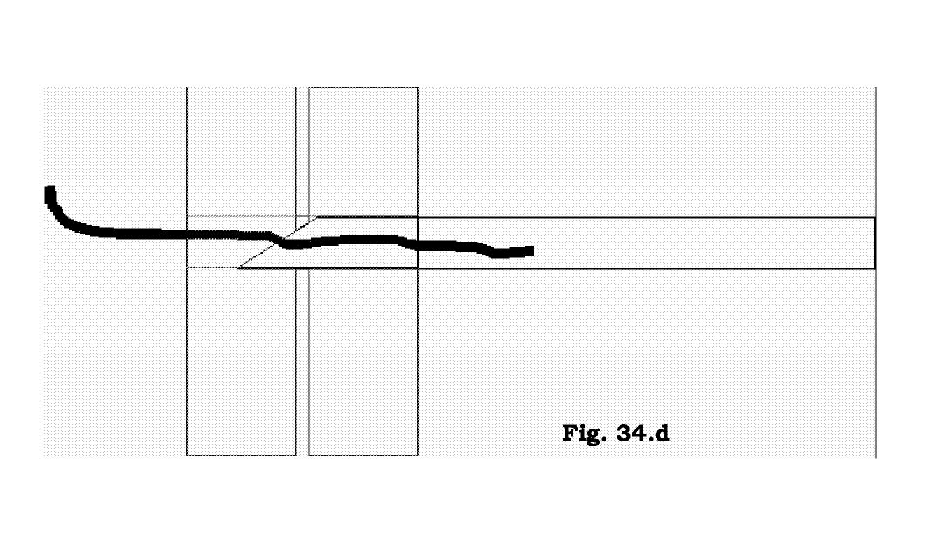

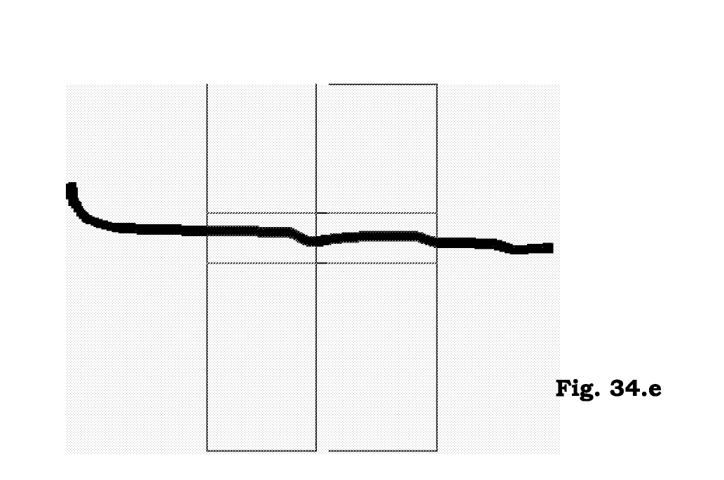

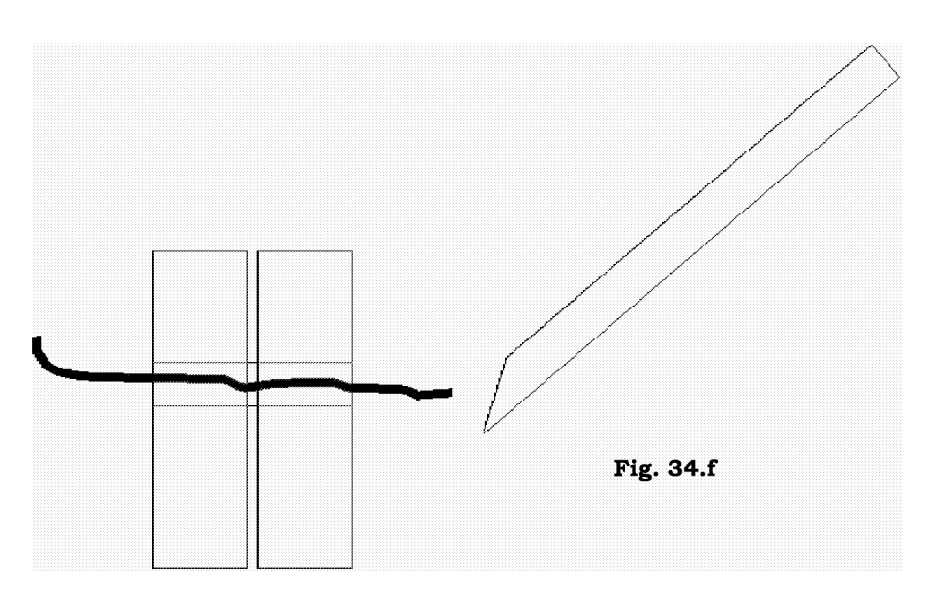

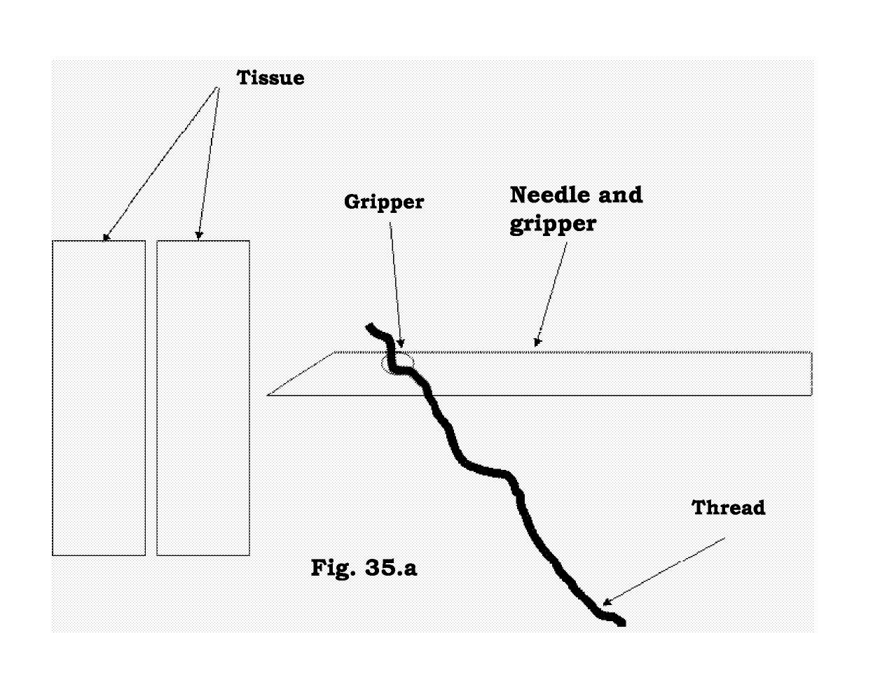

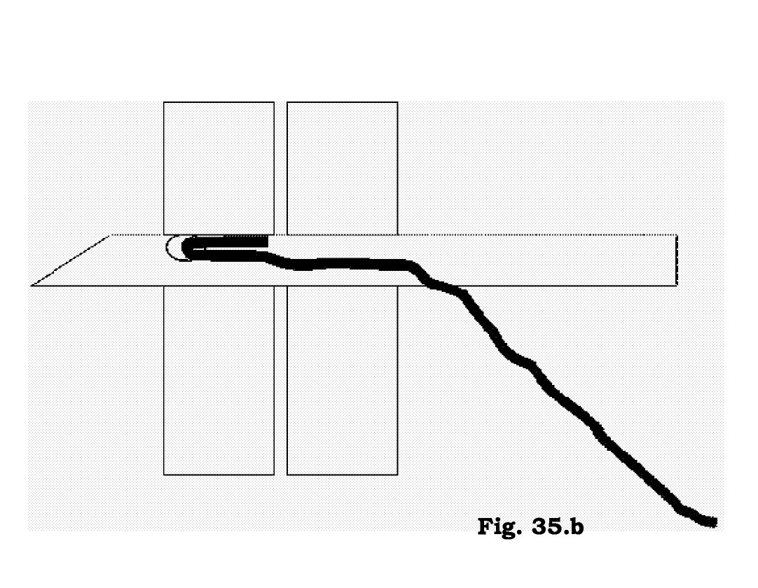

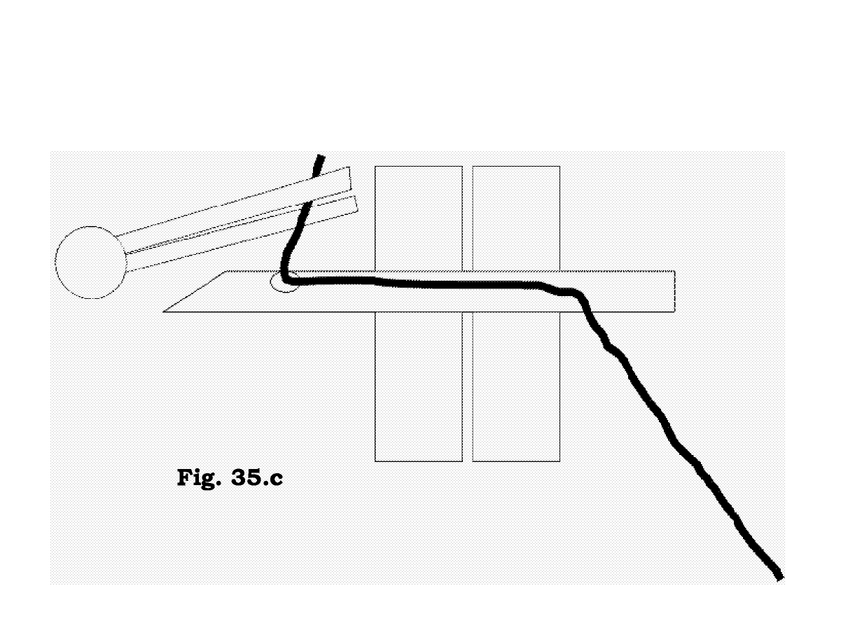

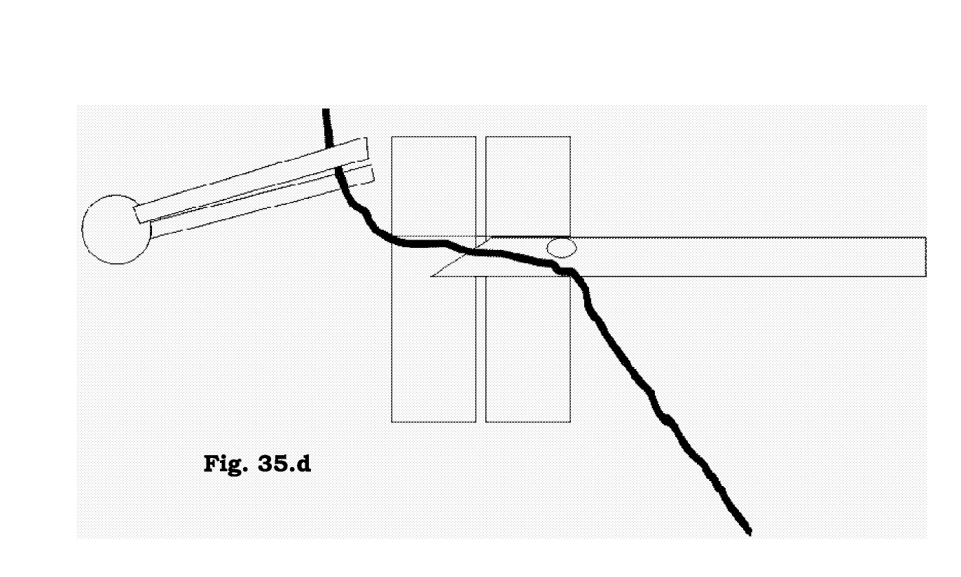

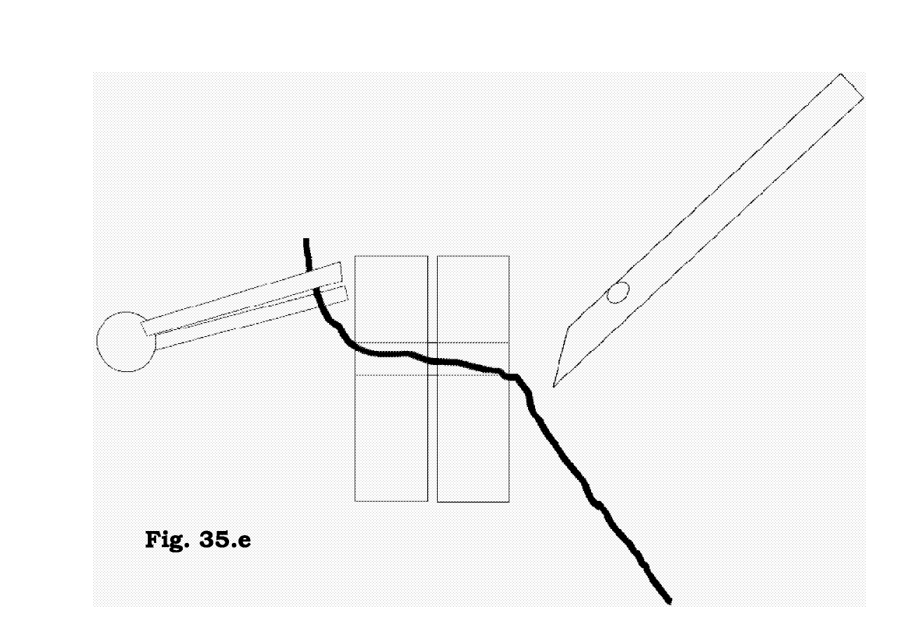

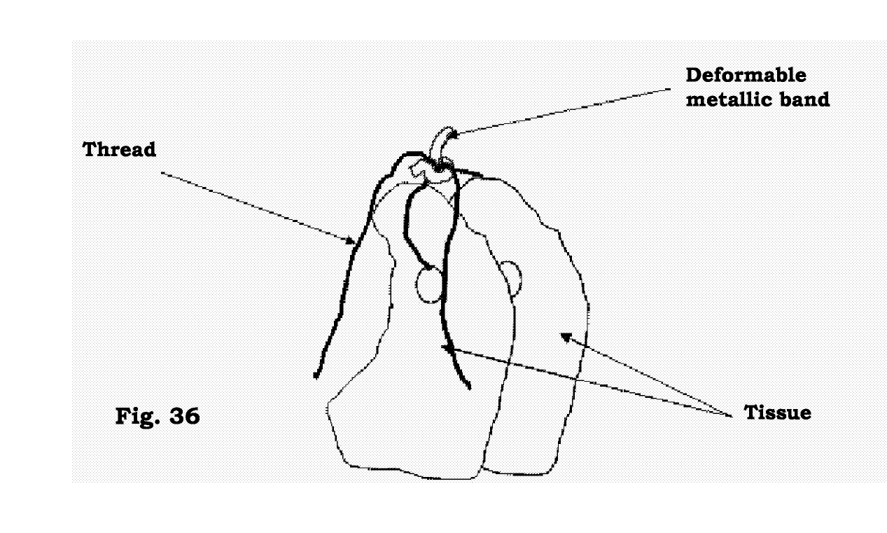

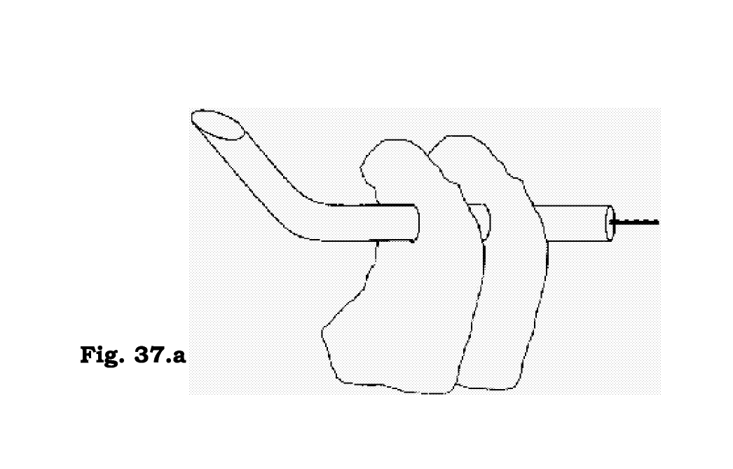

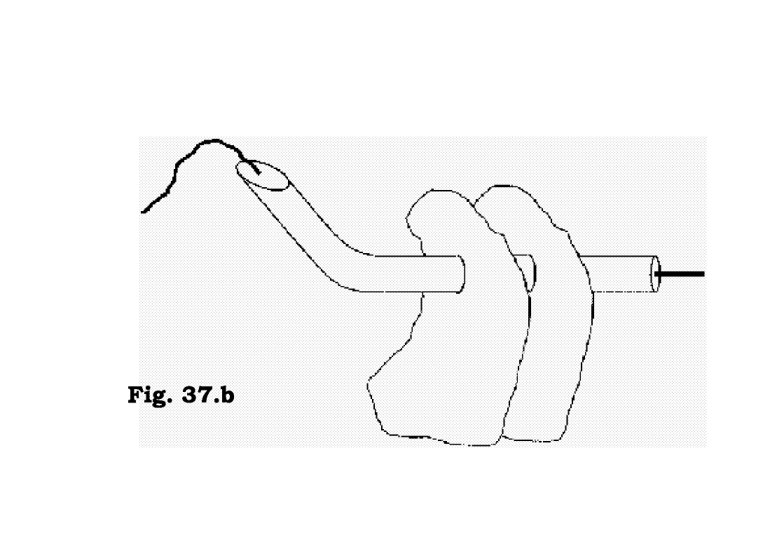

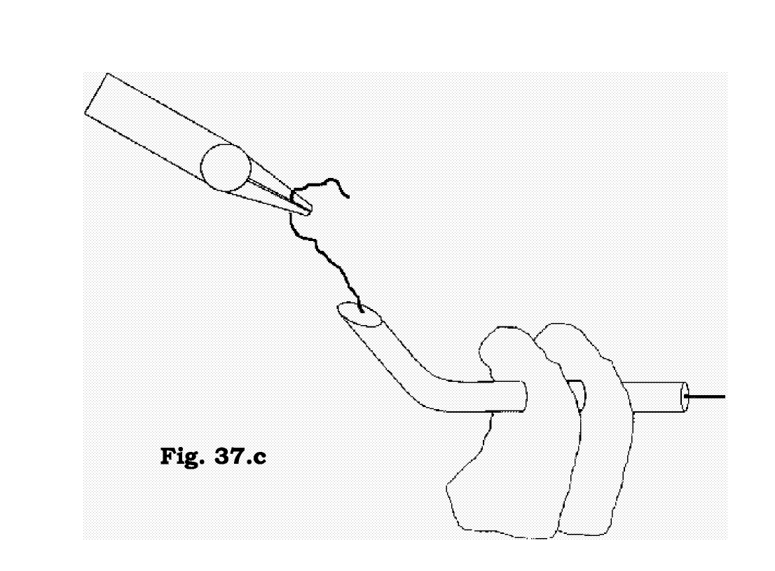

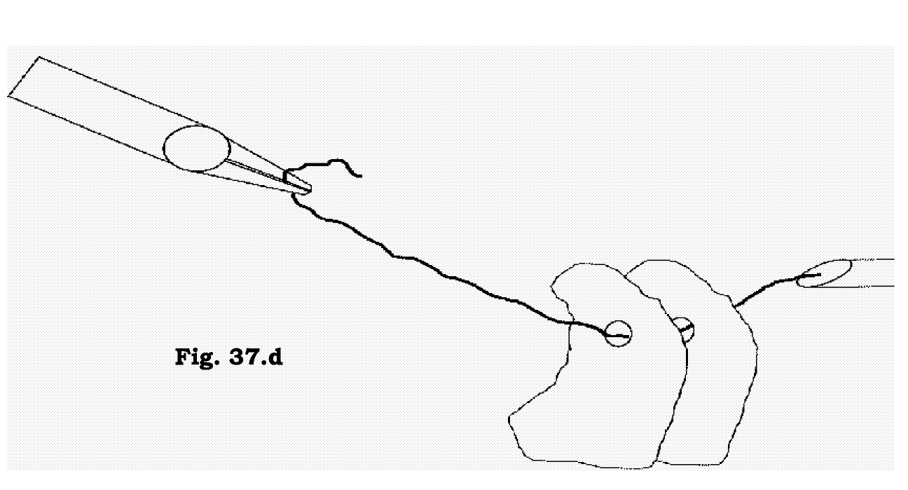

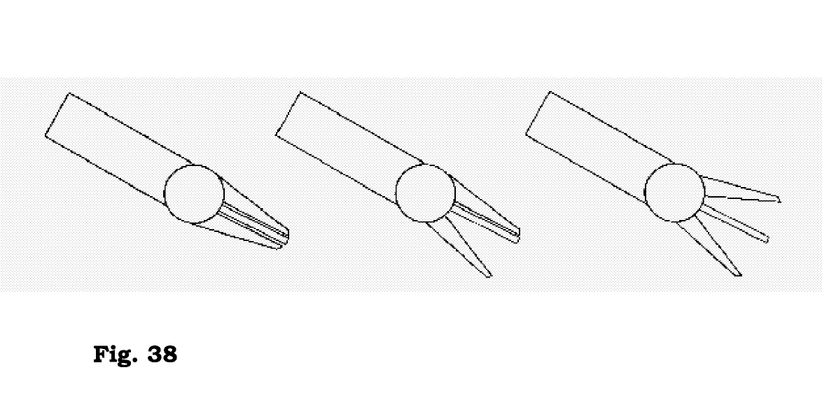

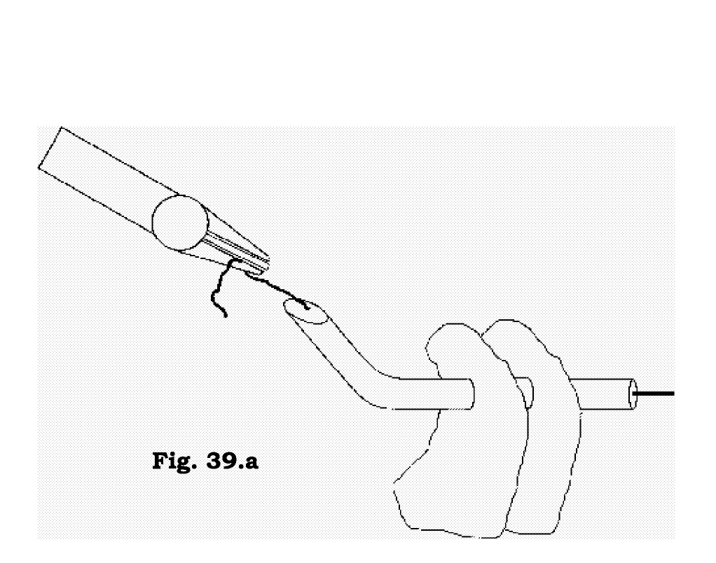

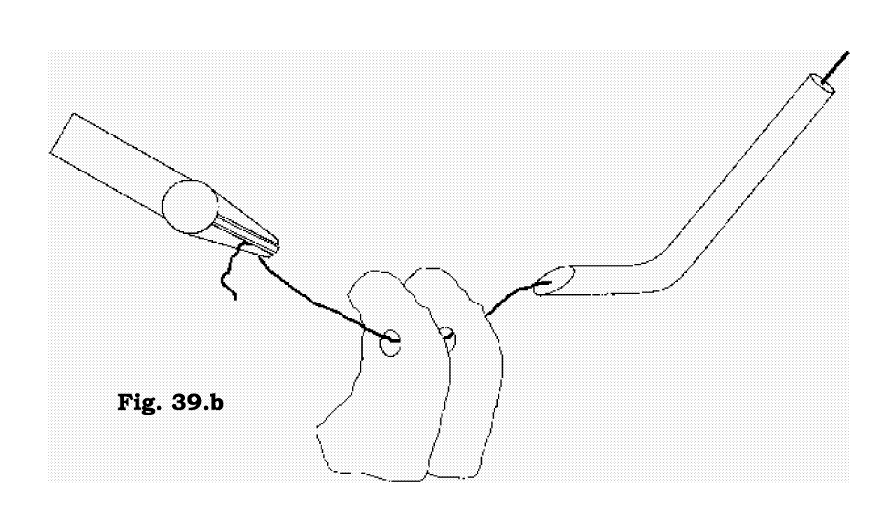

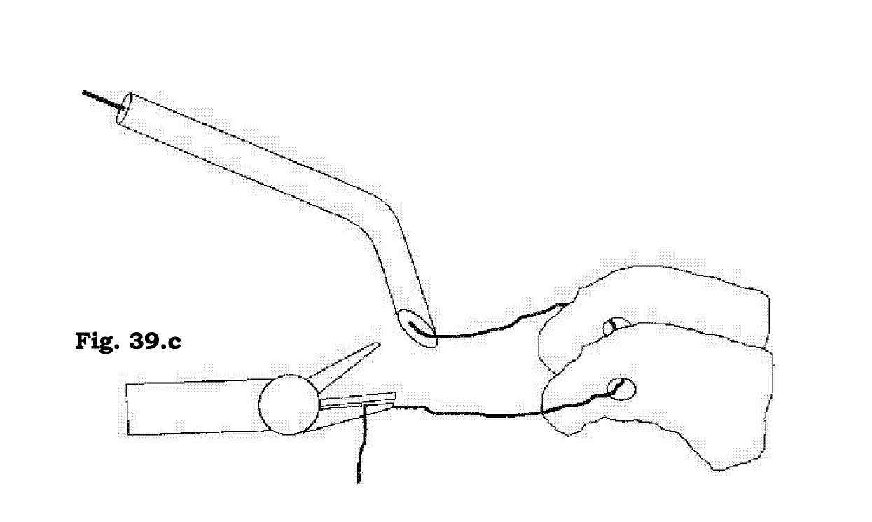

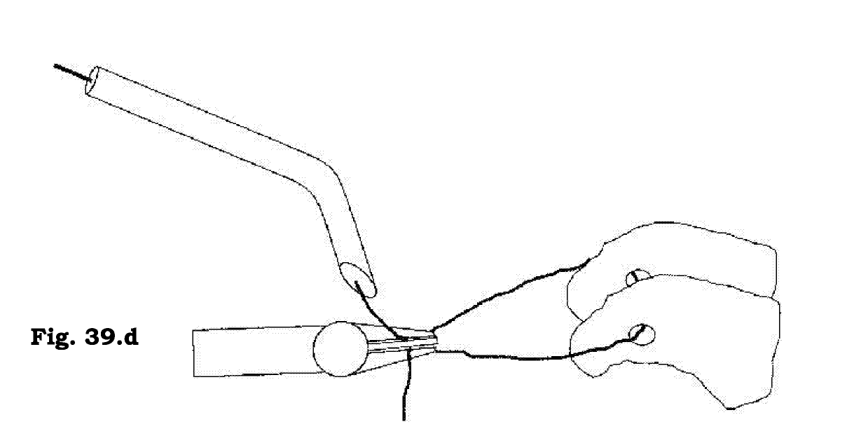

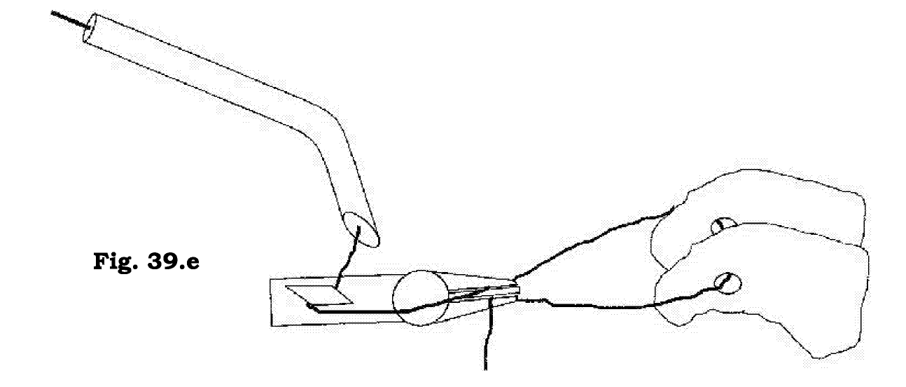

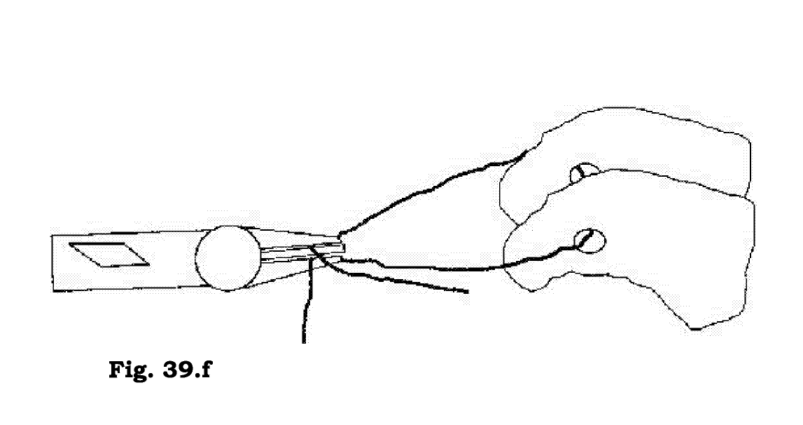

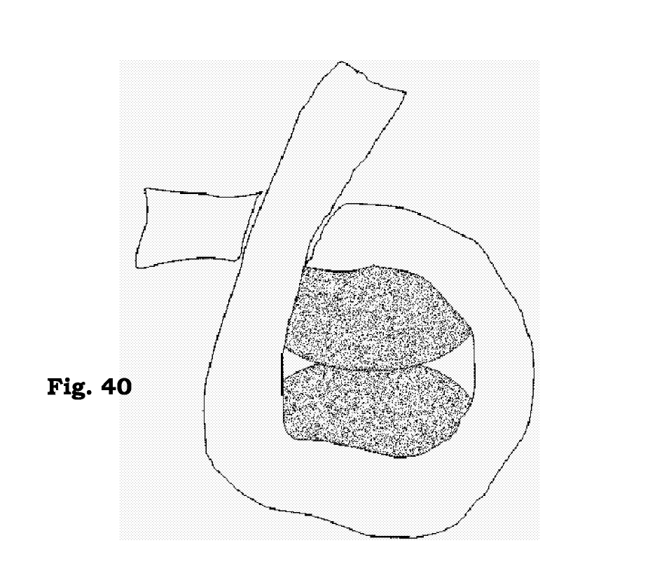

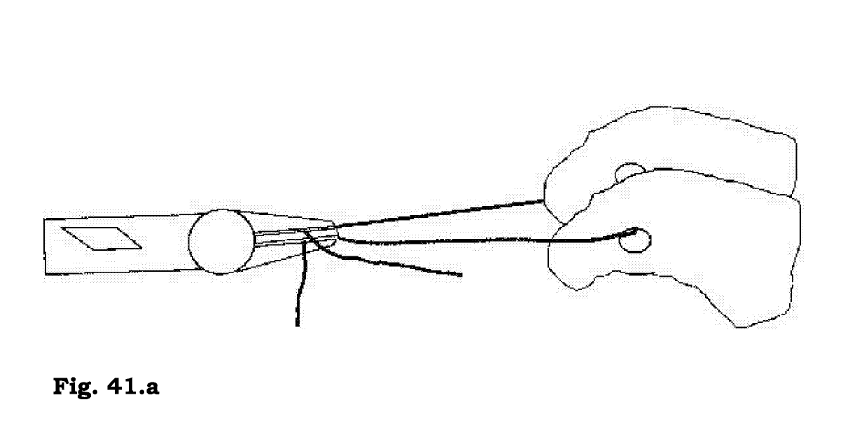

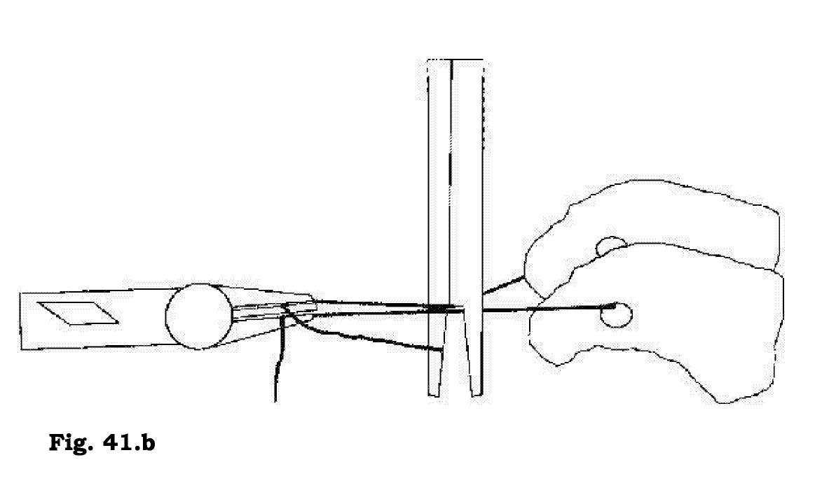

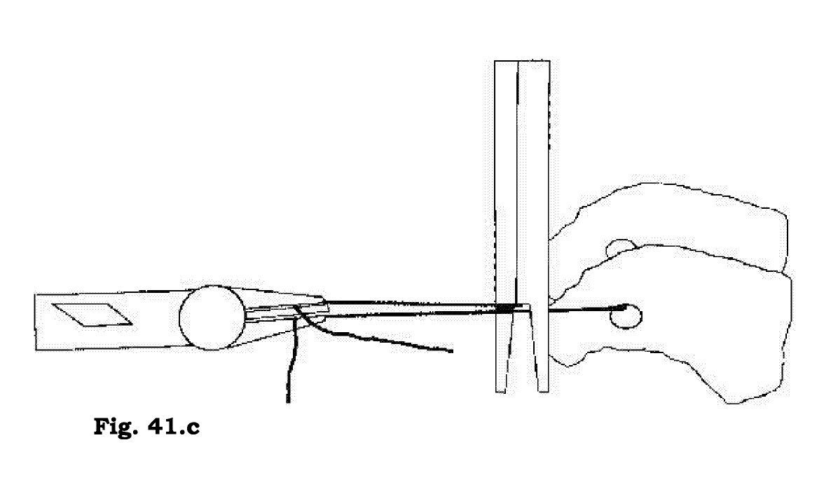

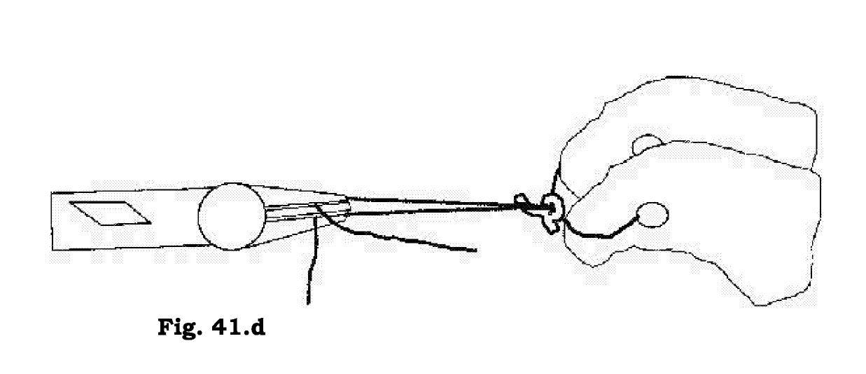

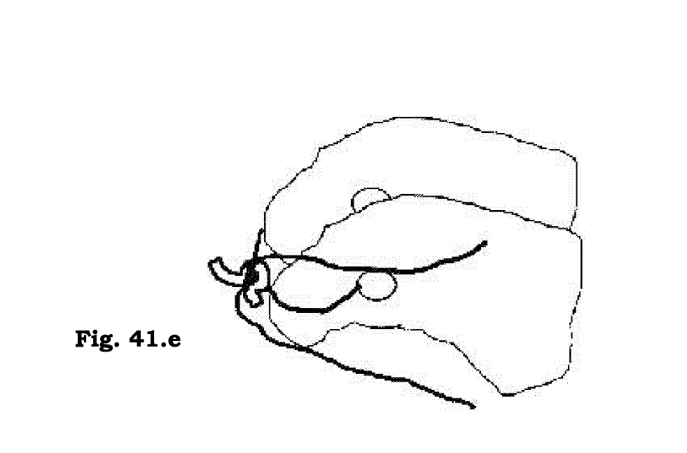

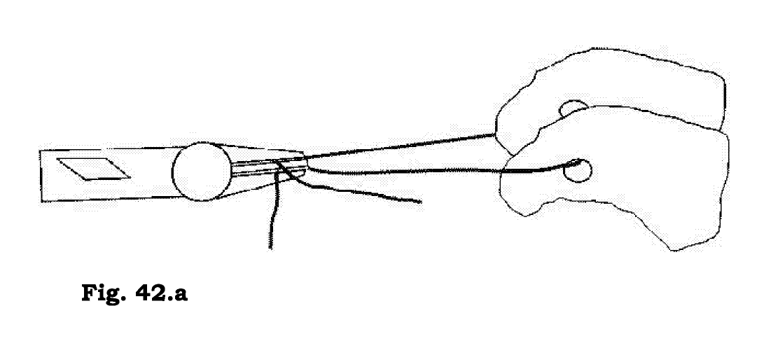

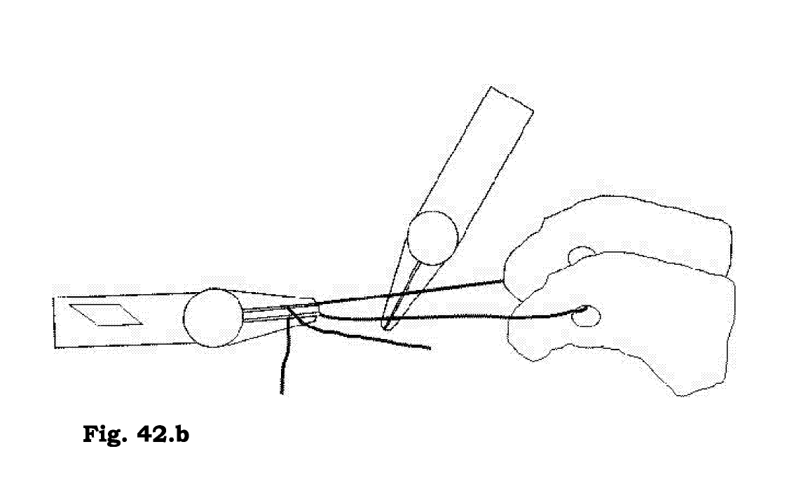

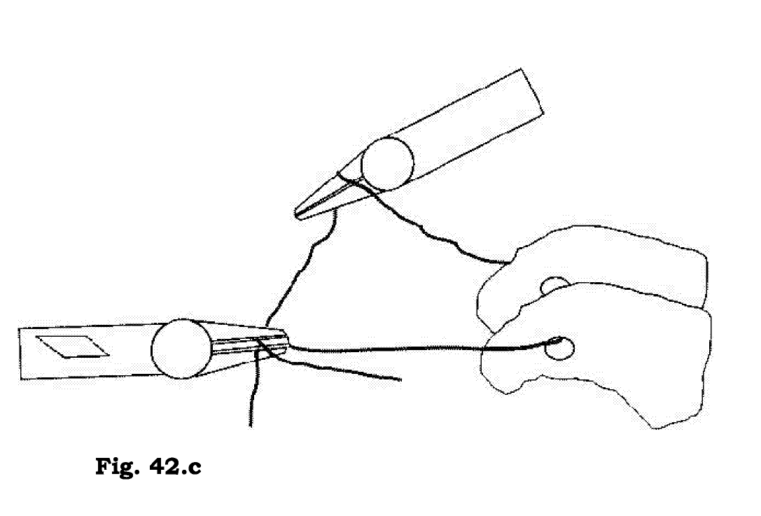

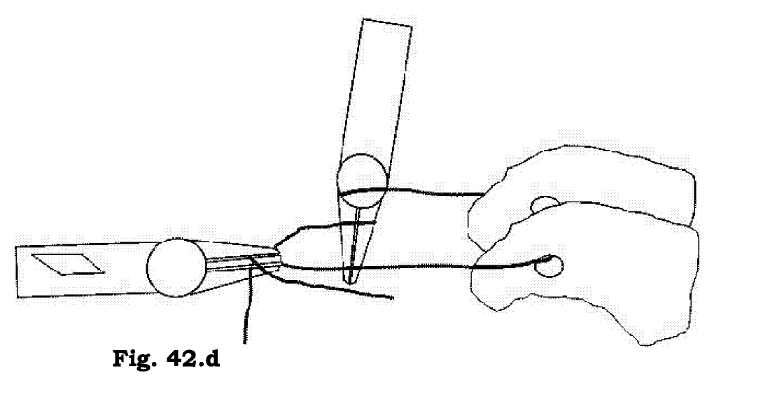

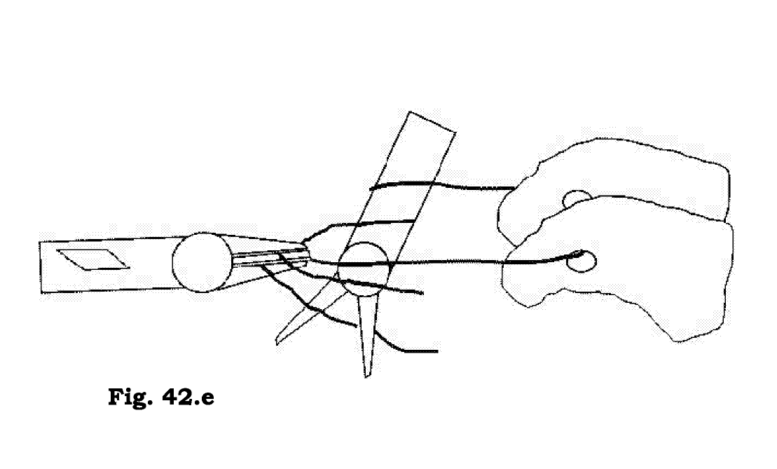

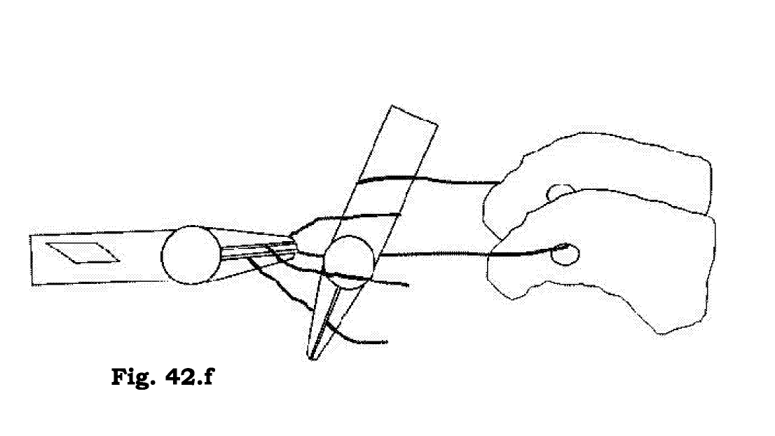

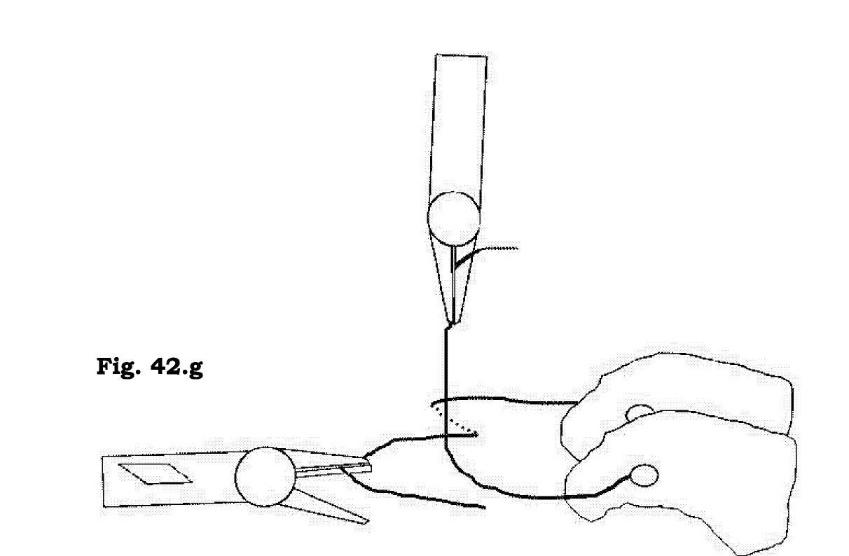

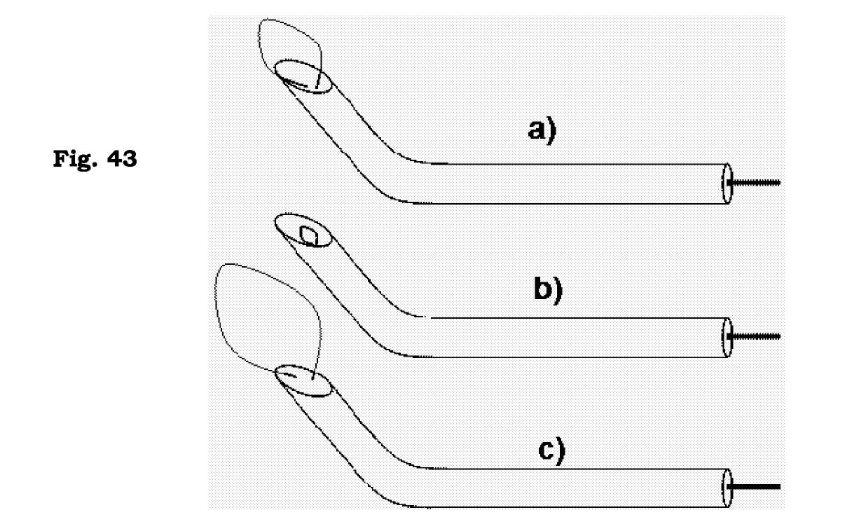

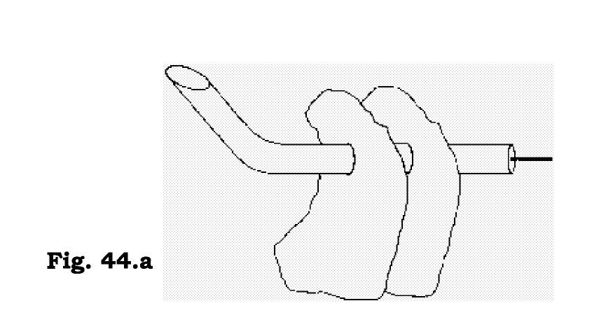

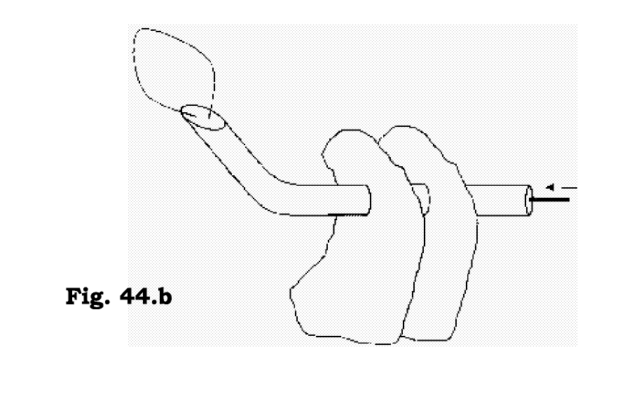

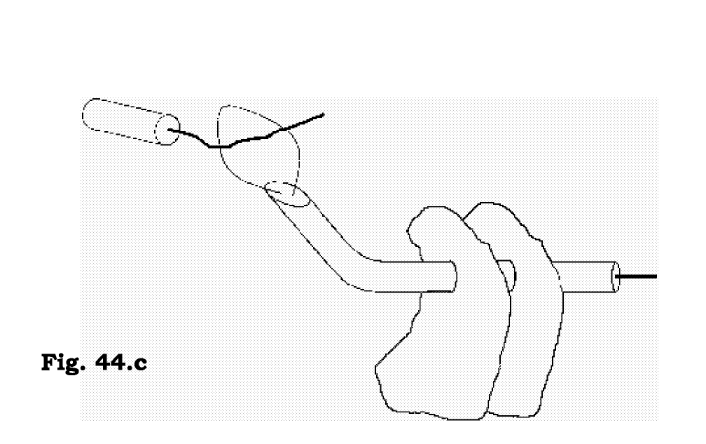

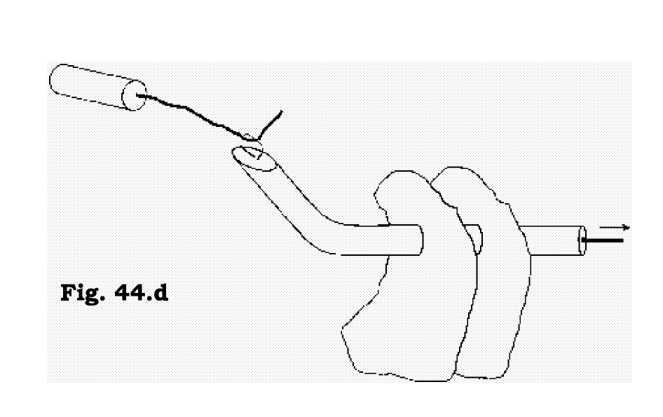

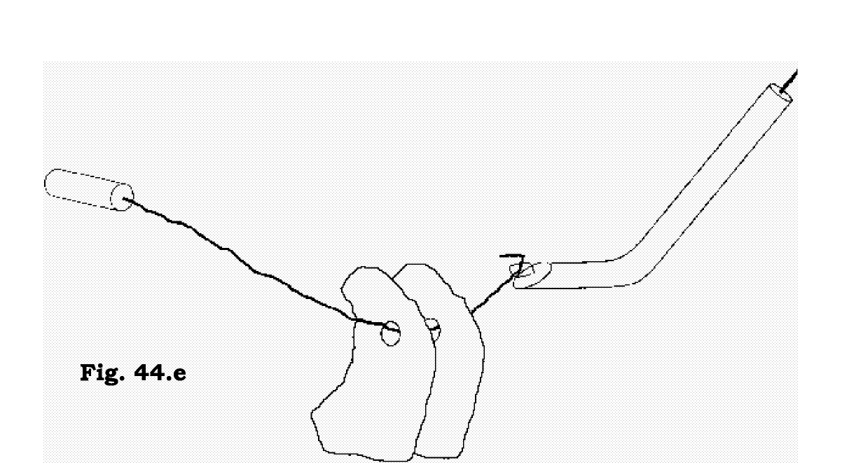

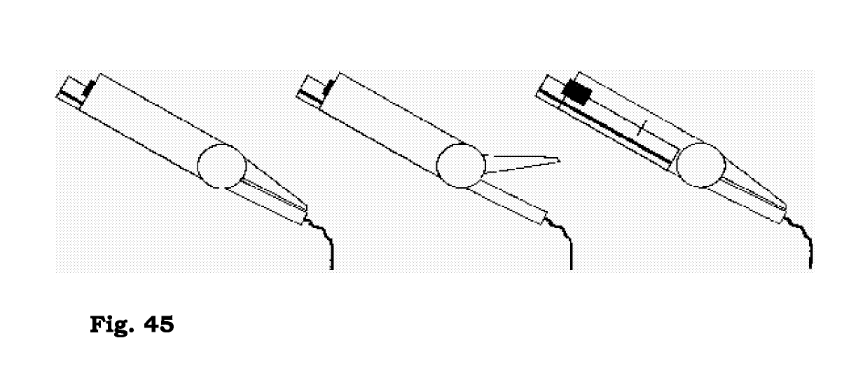

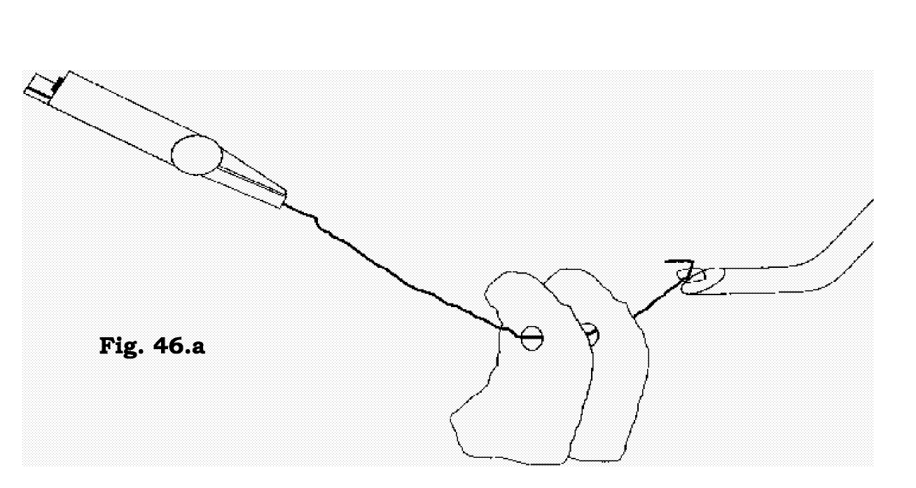

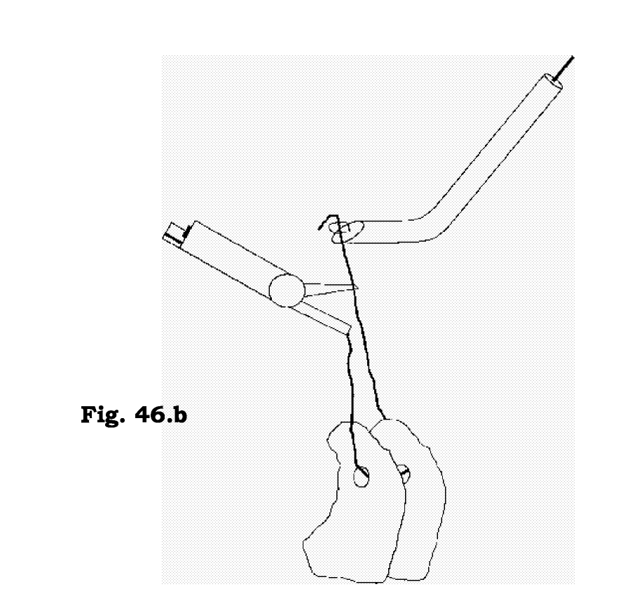

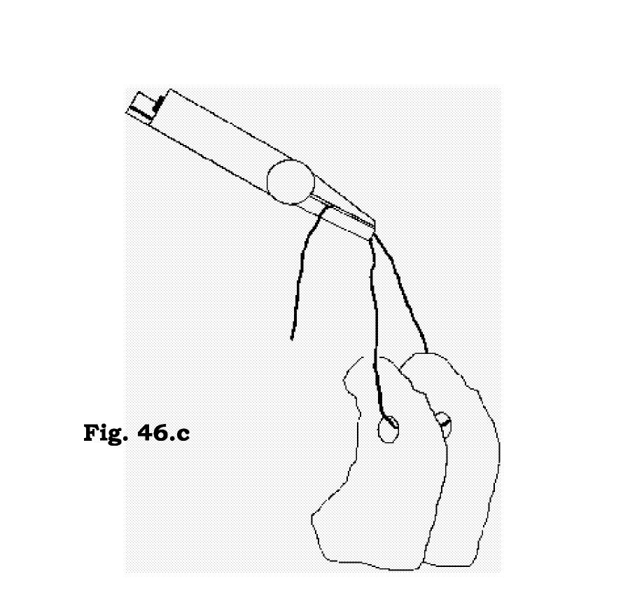

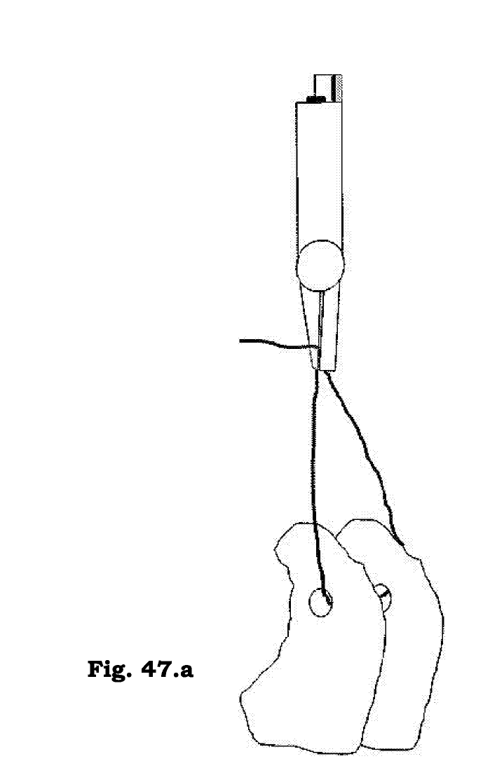

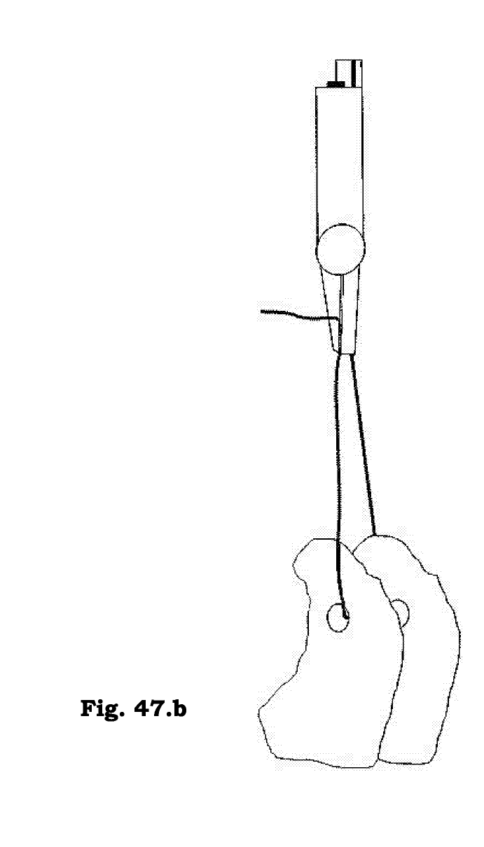

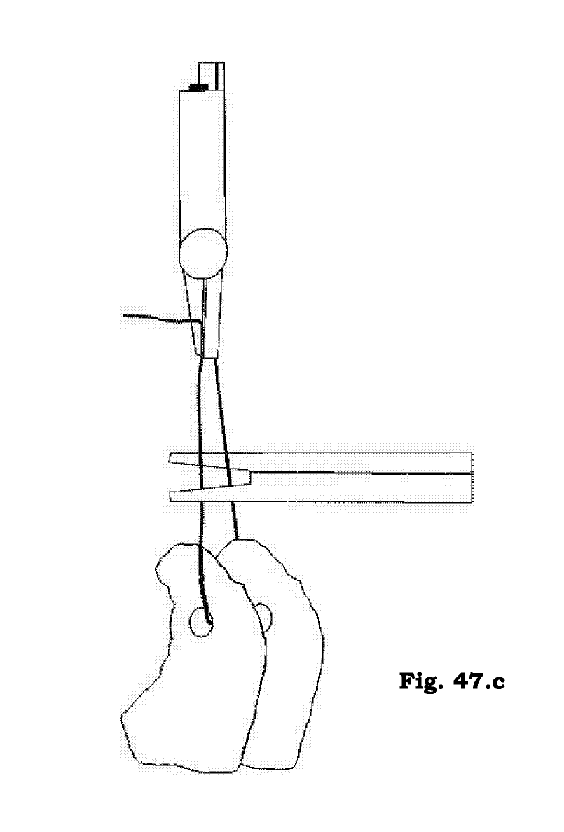

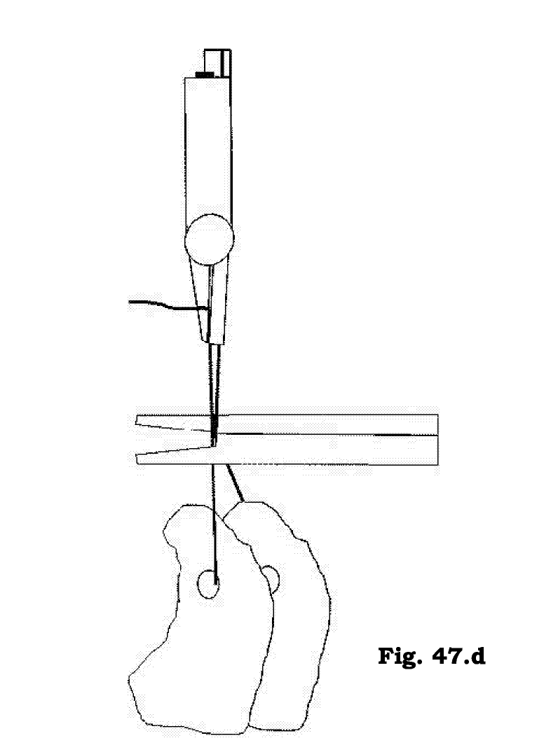

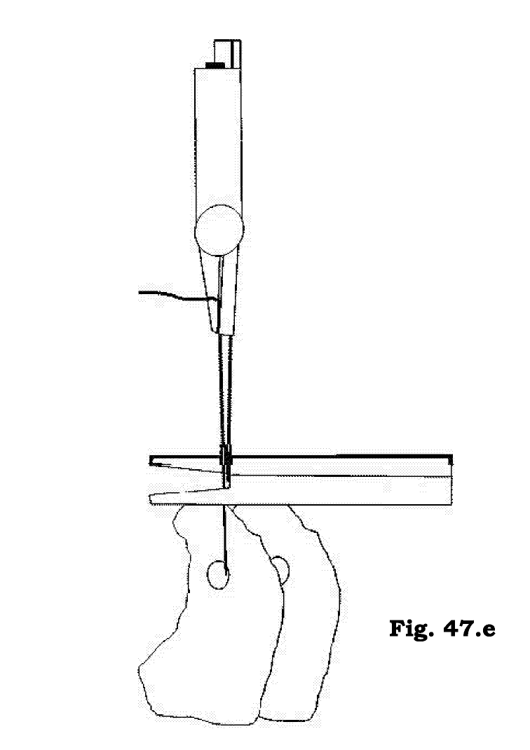

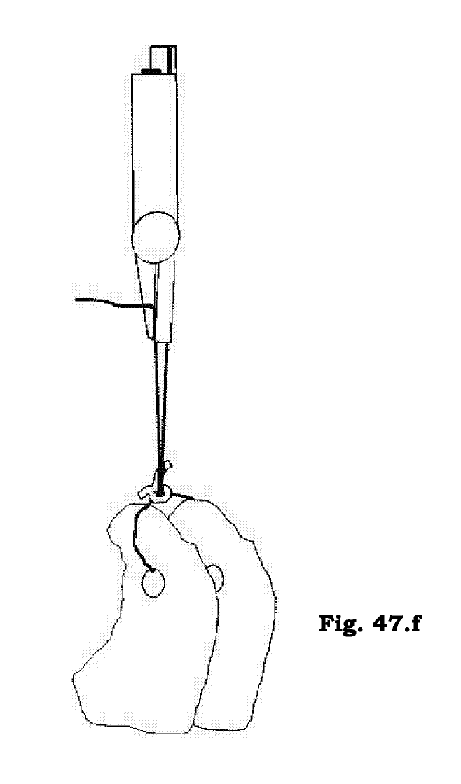

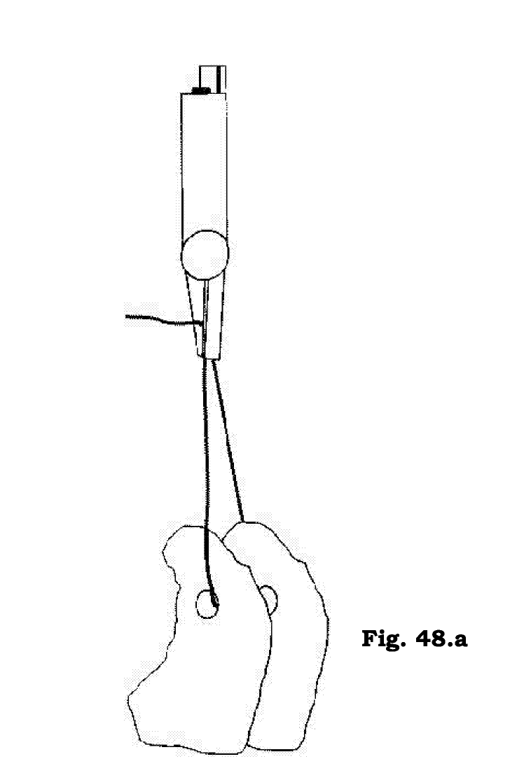

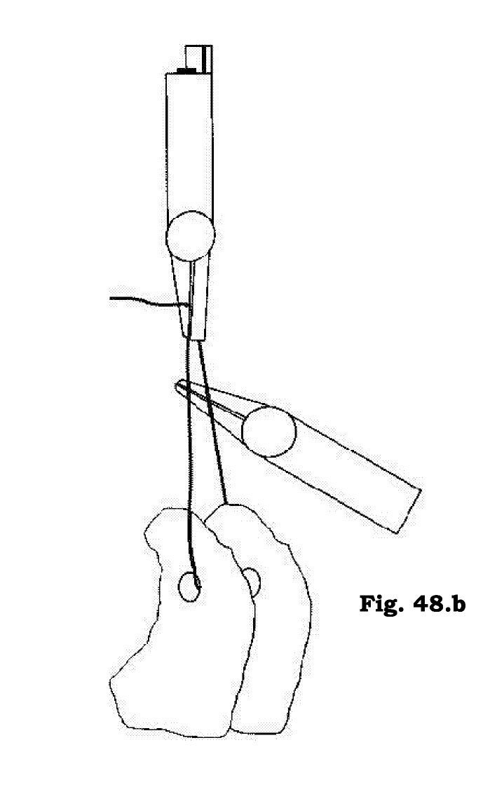

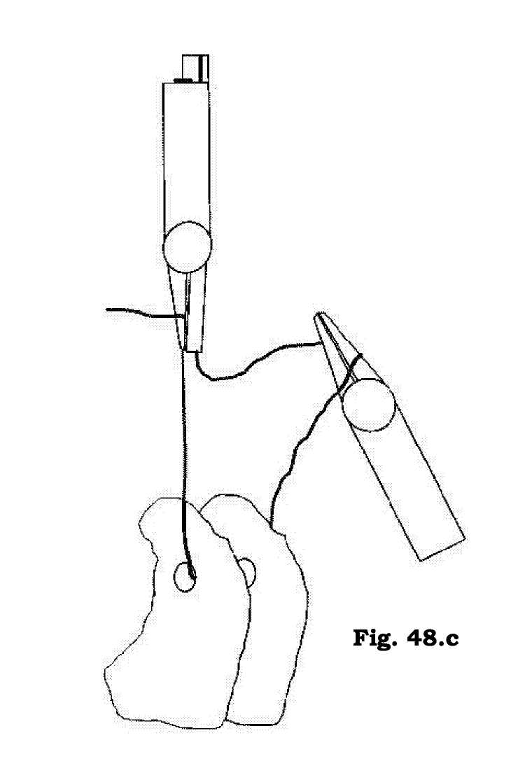

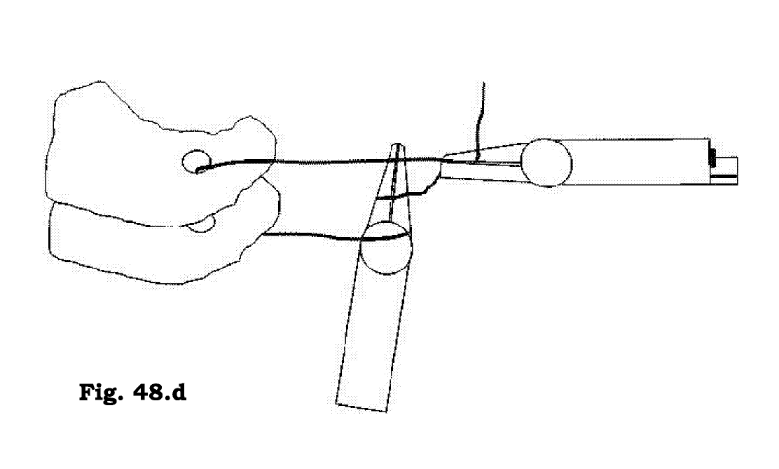

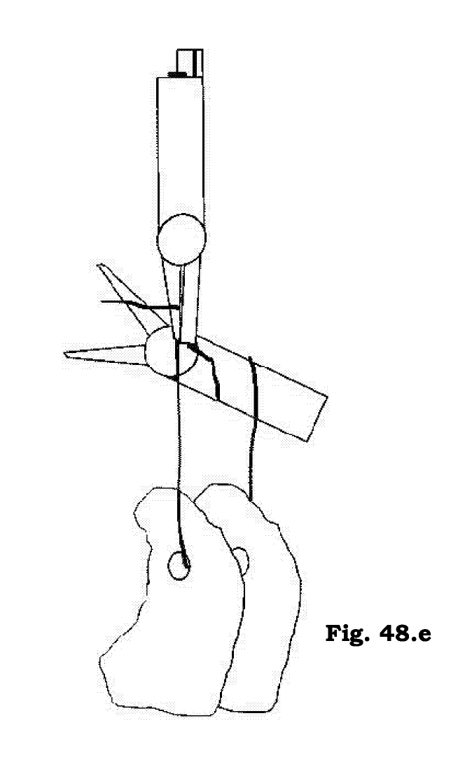

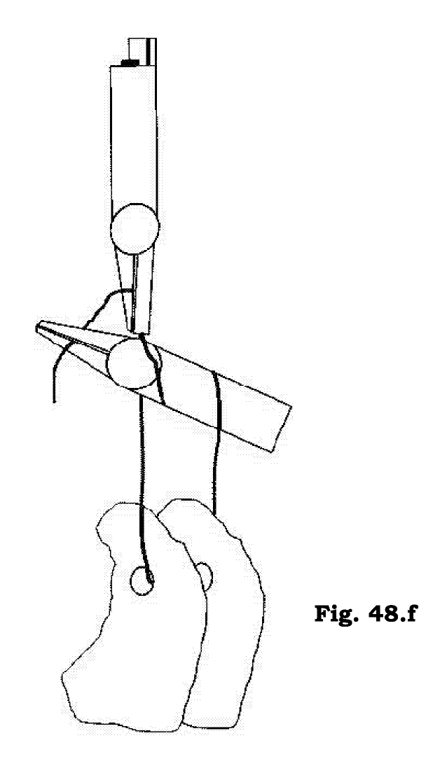

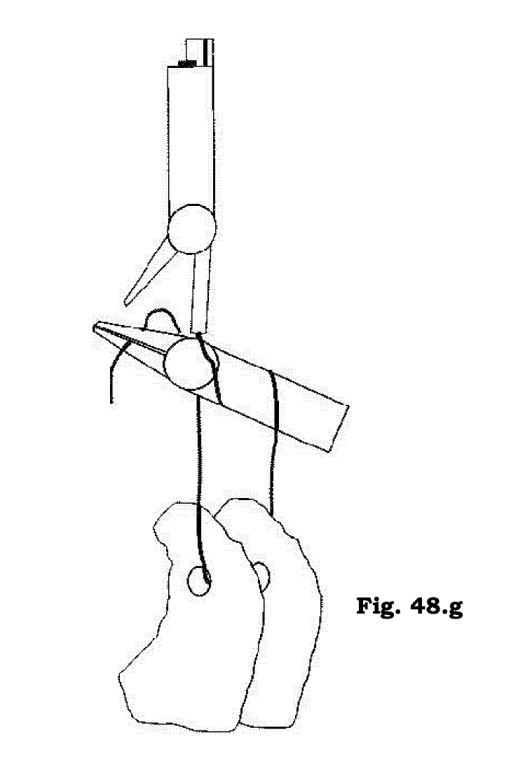

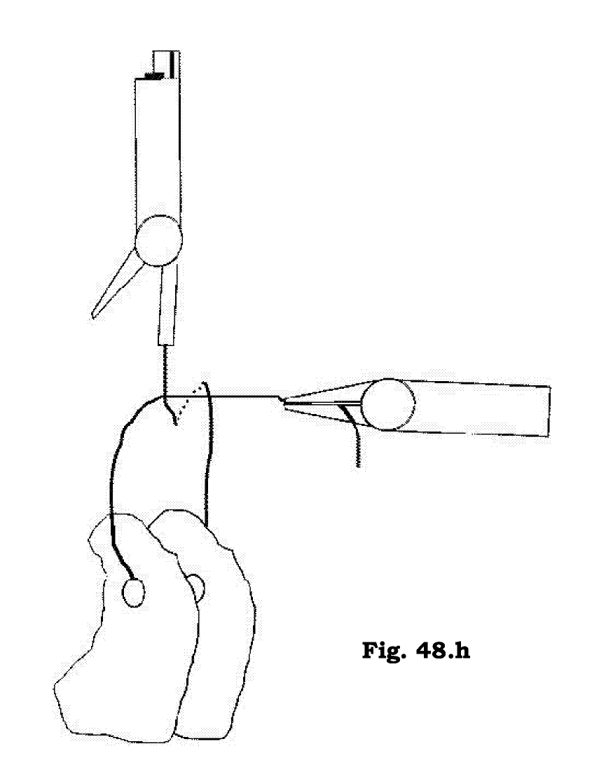

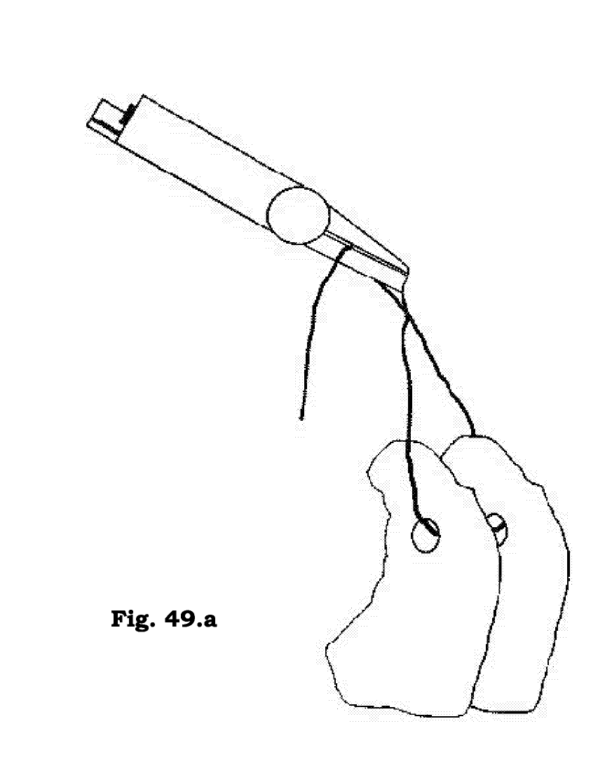

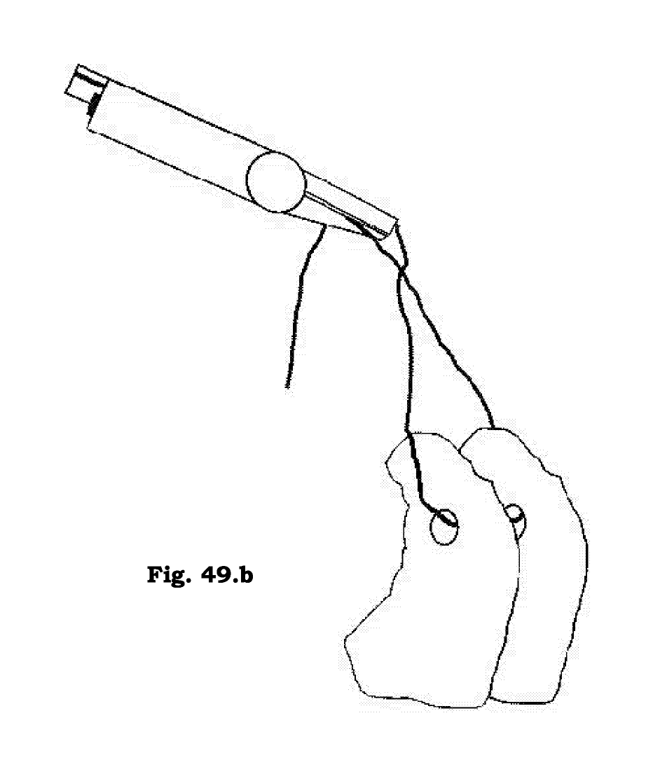

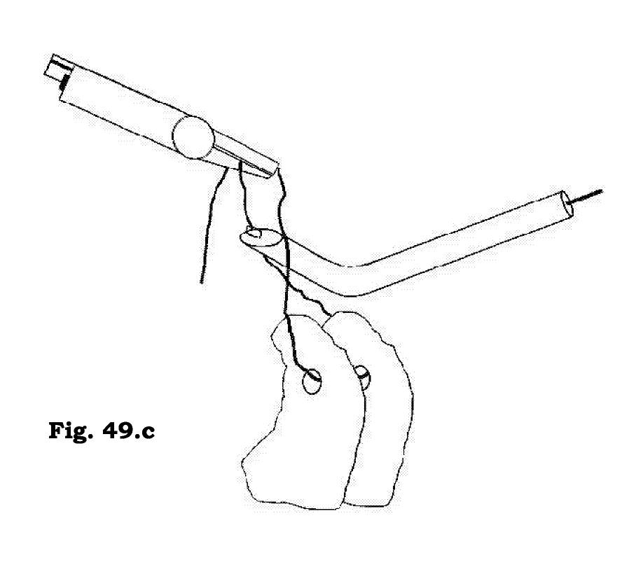

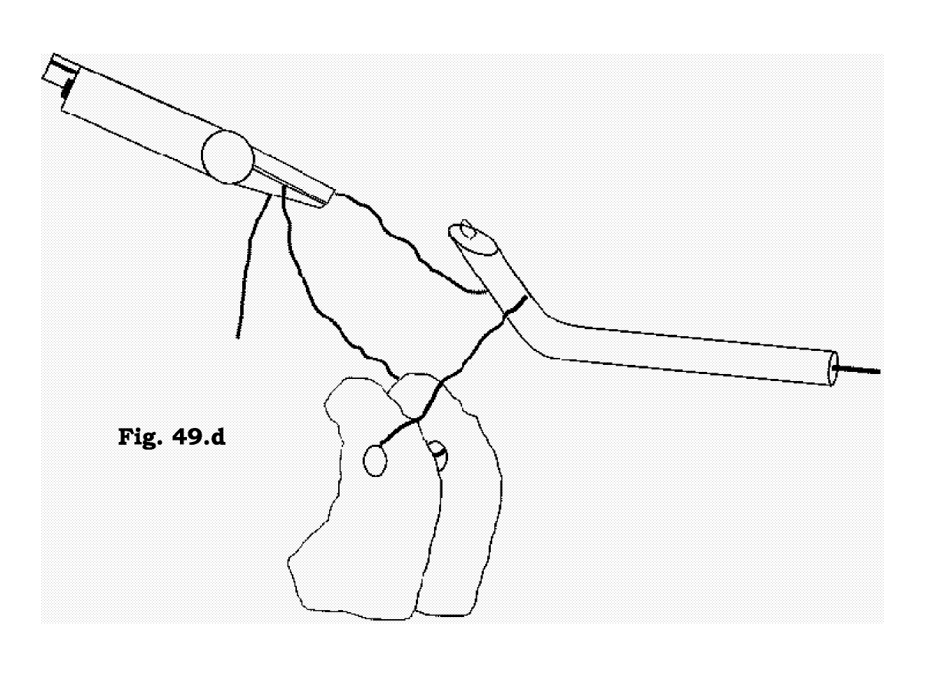

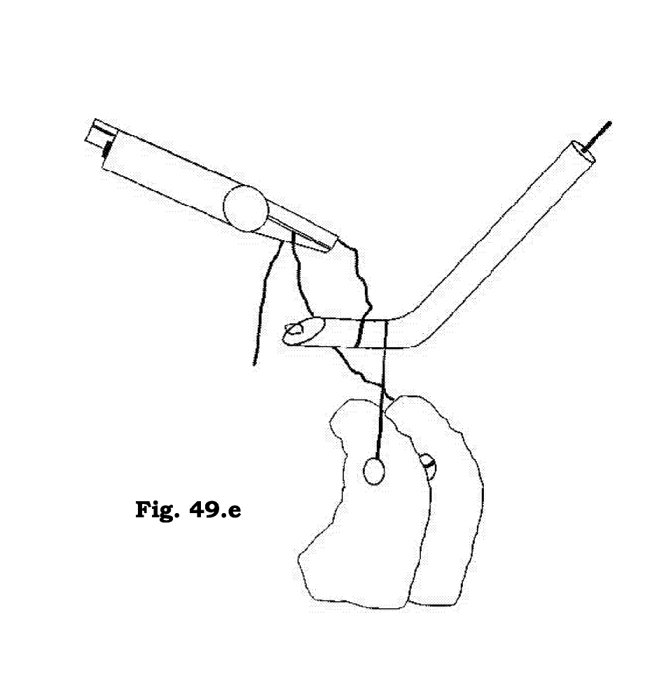

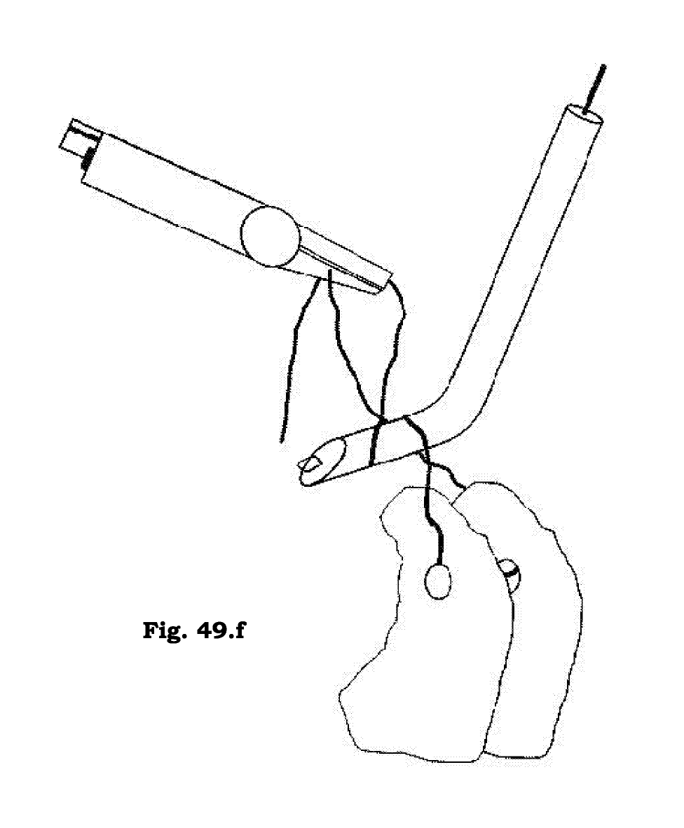

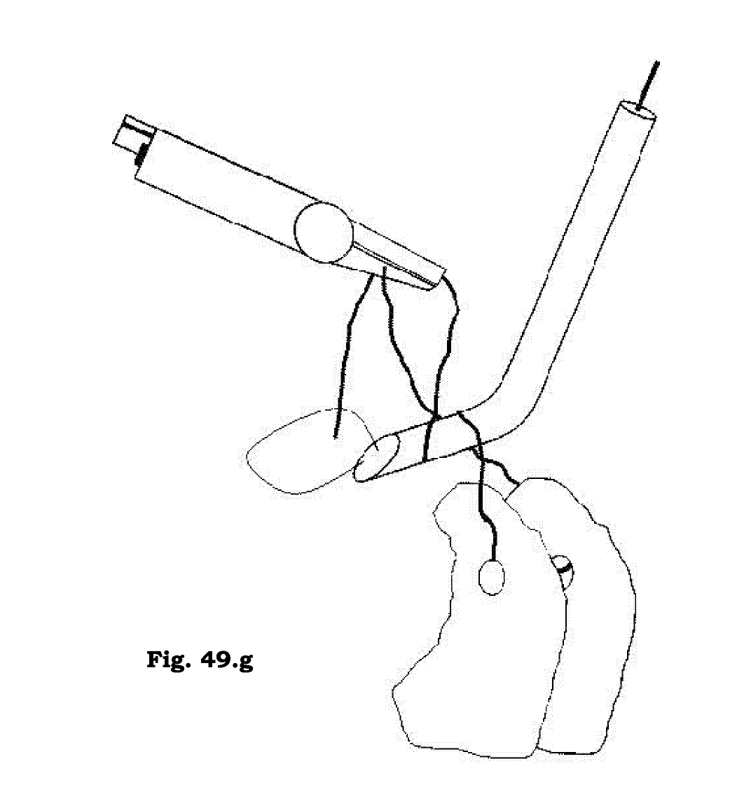

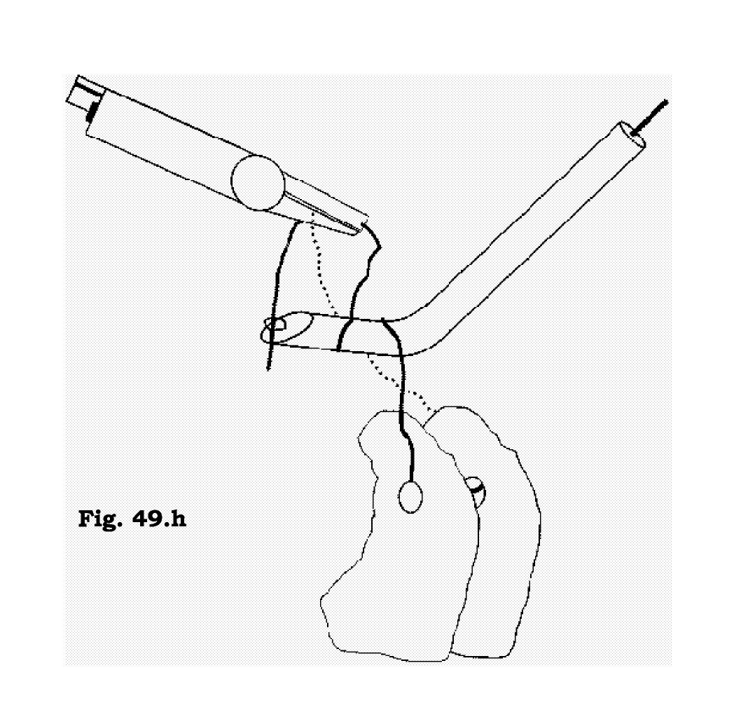

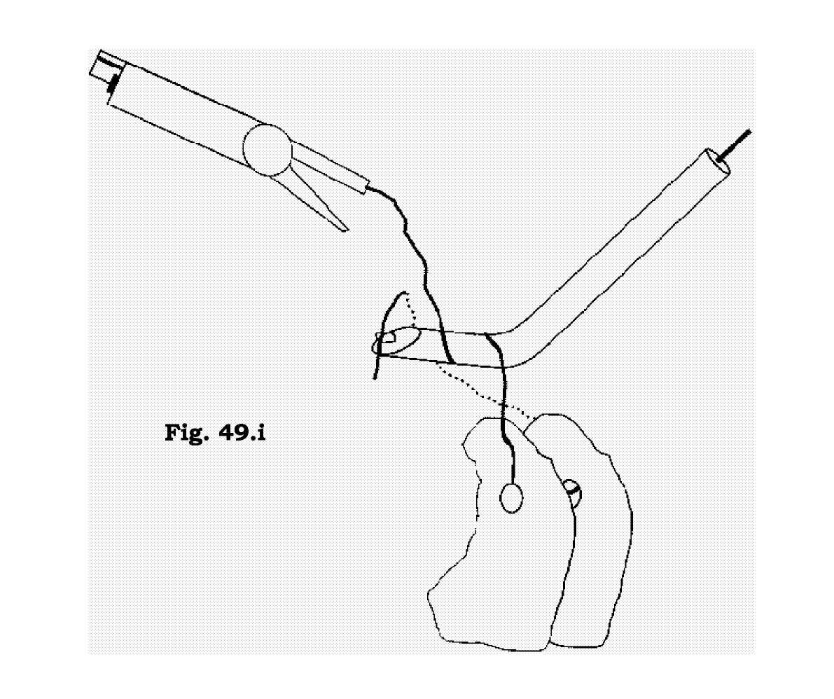

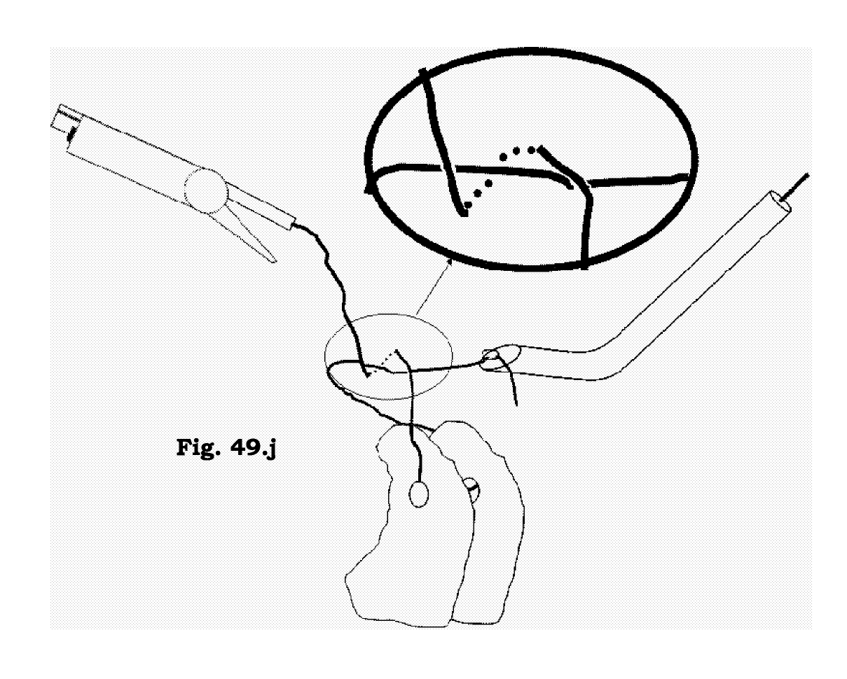

FIGS. 24 and 25 are taken from web cites on the Internet,

FIG. 26 shows the perspective view of laparoscopic clips instrument,

FIG. 27 shows an exploded view of the instrument of FIG. 26,

FIG. 28 shows a detailed view of a front part of the instrument of FIGS. 26-27,

FIG. 29 shows a further detailed view of the front part,

FIG. 30 shows a detailed view of components of the front part of the instrument,

FIG. 31 shows a detailed view of jaws used with the instrument of FIGS. 26-27,

FIG. 32 shows one jaw of FIG. 31,

FIG. 33 shows a detailed view of a clips magazine of the instrument of FIGS. 25-27,

FIG. 34 shows use of a needle as a channel for feeding a thread through tissues,

FIG. 35 shows the use of a needle with a grip device for feeding a thread through tissues,

FIG. 36 shows an alternative solution for joining the ends of a suture thread with a metallic band instead of a traditional knot,

FIG. 37 shows the use of a hollow needle with a device for feeding thread,

FIG. 38 shows a double forceps,

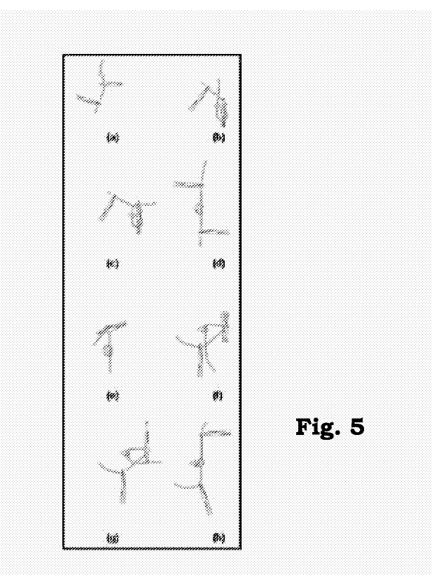

FIG. 39 shows the use of a double forceps according to FIG. 5 together with a hollow needle and a device for feeding thread,

FIG. 40 shows the use of a metallic clip for joining and holding the ends of a suture thread,

FIG. 41 shows fastening of thread with the help of a metallic clip and an instrument for it,

FIG. 42 shows knotting with the help of a double forceps,

FIG. 43 shows a hollow needle with a grip device in the form of a steel wire,

FIG. 44 shows the use of a needle with a grip device,

FIG. 45 shows a single forceps with an integrated device for feeding thread,

FIG. 46 shows the use of a single forceps with integrated thread feeder and a needle with grip device,

FIG. 47 shows joining together thread ends in an integrated single forceps and thread feeder with the help of a metallic band,

FIG. 48 shows knotting of a thread with an integrated thread feeder and single forceps and a laparoscopic dissecting forceps, and

FIG. 49 shows knotting with the help of a needle and a grip device.

### DETAILED DESCRIPTION OF THE PRESENT INVENTION

### The Laparoscopic Instrument of the Invention

The below described instrument is only a suggestion of use of the principles of the invention in a complete instrument, other designs that utilize the same principles but in another way may be used and be possible solutions when it comes to designing a laparoscopic instrument.

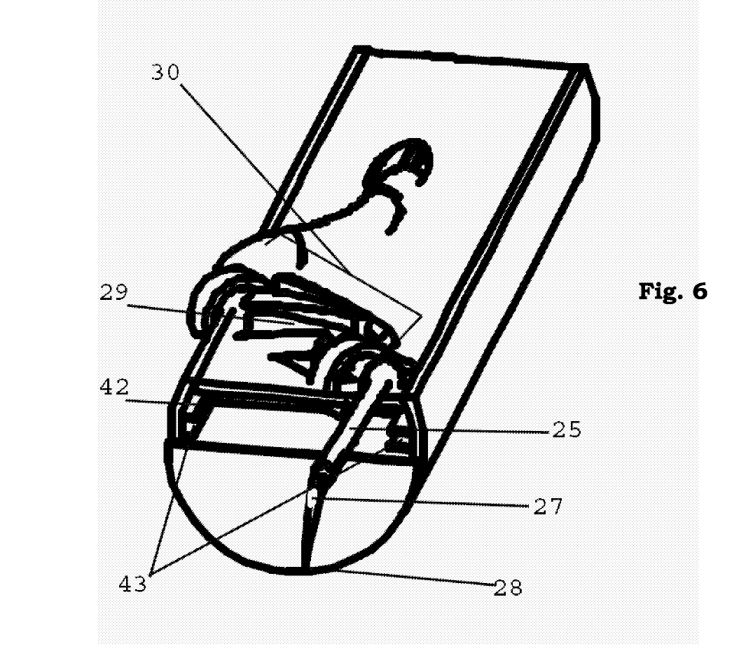

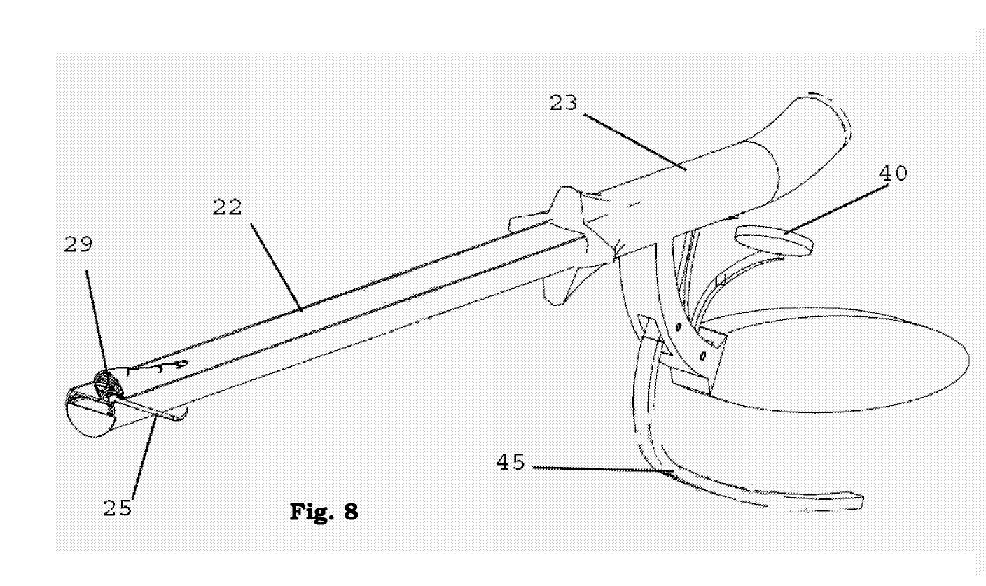

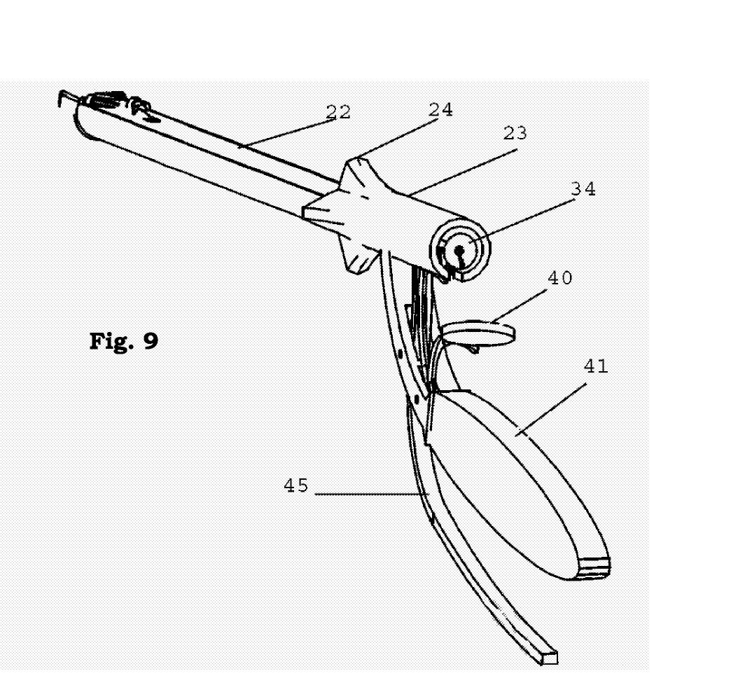

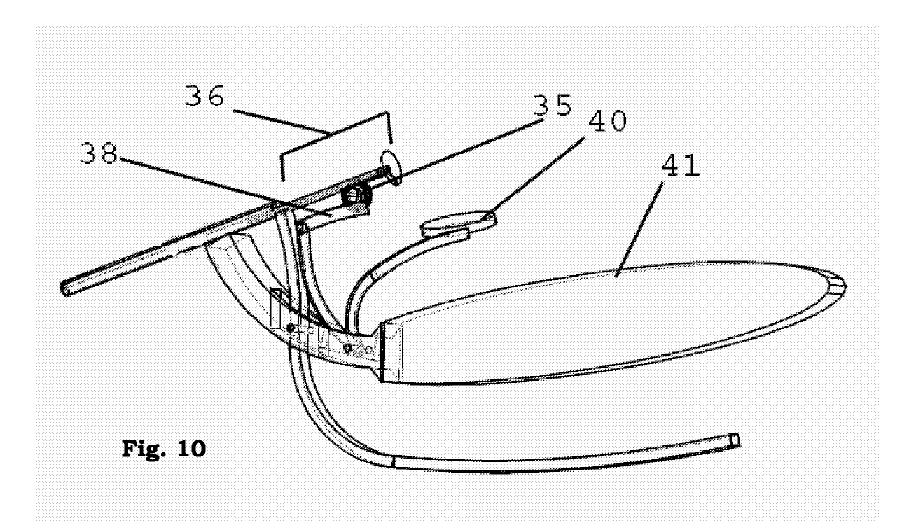

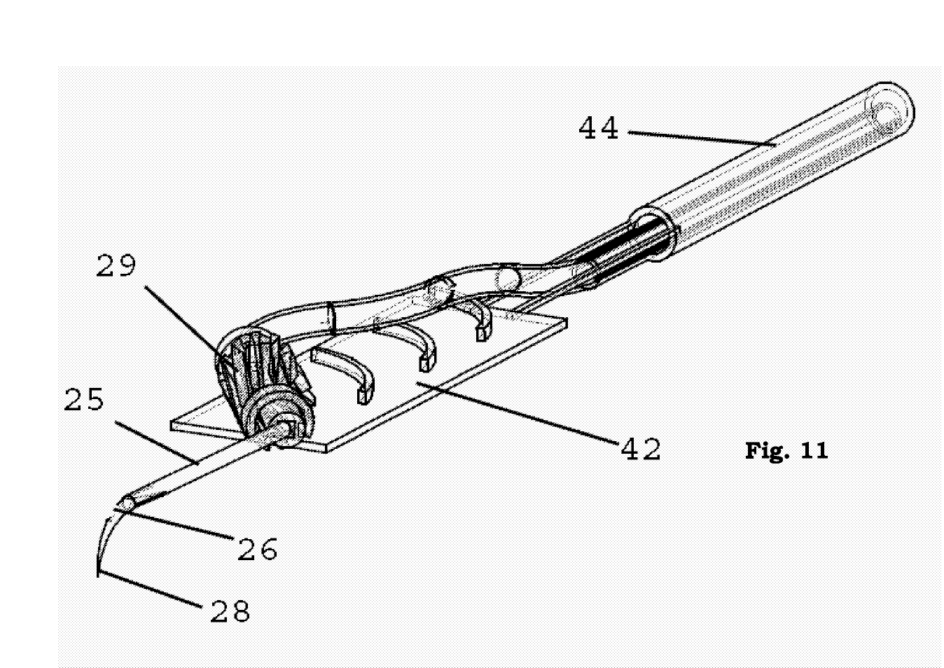

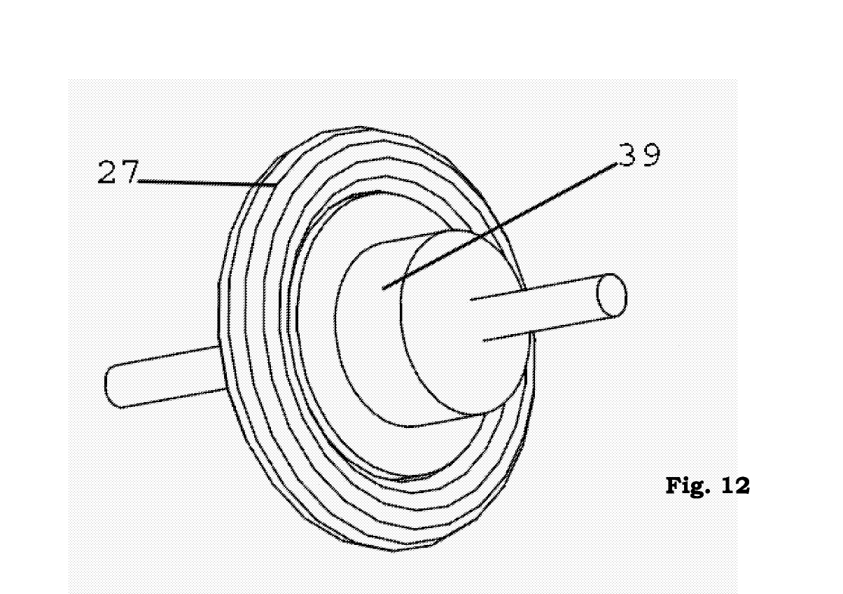

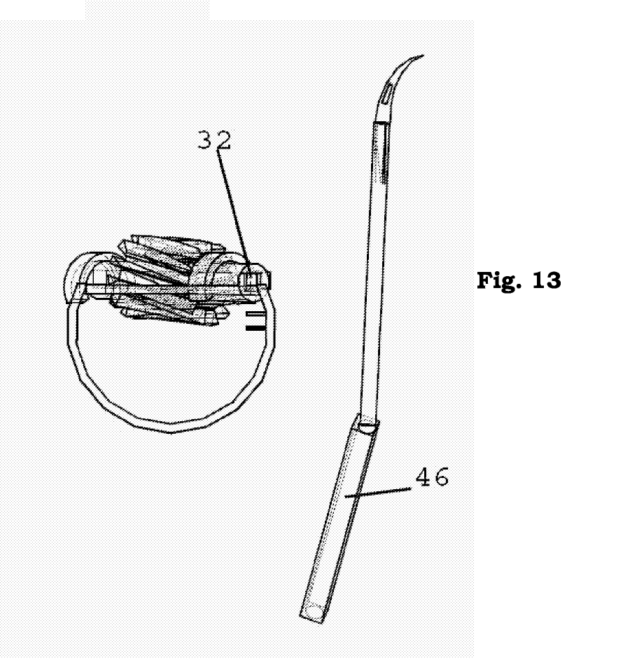

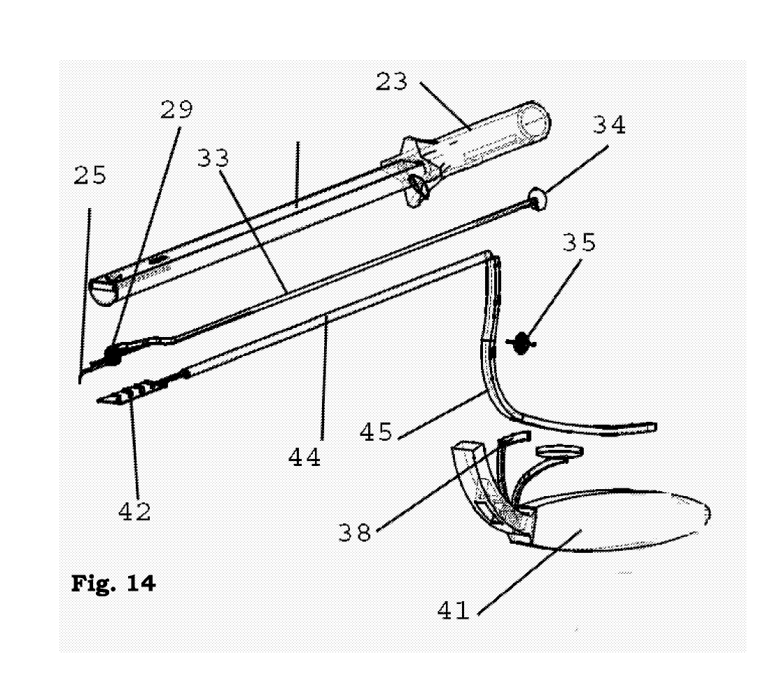

A laparoscopic suture instrument according to the present invention is shown in its entirety in FIGS. 6 to 14. The instrument consists of a metal shaft 22 having a half circular cross-section. The shaft 22 is about 30-35 cm long and has a diameter of about 1 cm. The shaft 22 may be entered into laparoscopic ports with diameters of 13 mm. The instrument also displays a handle 23 which is mounted on one end of the shaft 22 and has a rotational device 24 in order to be able to rotate the shaft 360 degrees when needed during suturing.

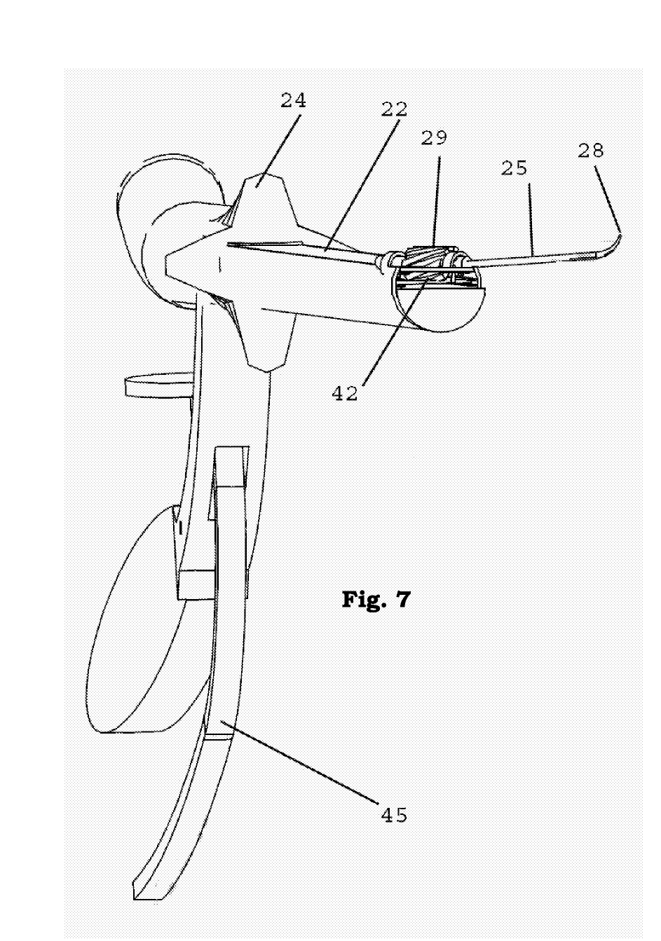

A needle 25 is mounted on the other end of the shaft, that can be used for obtaining a channel when the needle is entered into tissues that are to be sewn together. The needle is bent at its tip 28, whereby the bending angle can vary depending on the operation and the tissues that are to be sewn together. The needle is also characterised in that it is hollow along its entire length. An operation thread or a metal thread can be fed in via this hollow channel and may exit through an opening 27 at the outer side of the needle 25, which opening is about half a centimetre proximal of the tip of the needle 28. The needle 25 is mounted on its proximal end with a rotational cog wheel cylinder 29, which is rotated 180 degrees around its longitudinal axis 30 in order to turn the needle from a passive position FIG. 6, with the tip of the needle in a backwards position to an active position, FIG. 7, with the tip of the needle in a forward direction for penetration of tissue, whereby the latter position is suturing position.

The cog wheel cylinder 29 is hollow along its longitudinal axis. A pipe 31, suitably made of a plastic material and which is exchangeable, is connected between the channel 32 of the cog wheel cylinder and a channel 33, that is present along the entire shaft 22 and the handle 23 and protrudes via a trough-like opening 34. Through this opening 34 the operation thread or the metal thread is fed, the thread is then fed further through the whole metal channel 33, the plastic pipe 31, the hollow needle 25 and into the body through the needle opening 27.

A thread feeding device 35 is arranged, which is mounted in connection to the proximal end 36 of the metal pipes that is in the handle 23. The thread feeding device 35 consists of two wheels, a larger wheel 37, that consists of metal and rubber, where the aim of the wheel is to feed the thread when it is rotated via a linearly movable device 38 that moves forth and back below and in contact with a smaller wheel 39 of the thread feeding device 35. A button 40 is arranged mounted in the part 41 of the handle 23, that is gripped. This button controls the forward and return movement of the linearly movable device 38.

The cog wheel cylinder 29 together with the needle 25 is rotated with the help of a metal plate 42, which is mounted inside the metal shaft 22 and which moves within a groove 43 in the shaft. The metal plate 42 is toothed on its upper side, whereby its curved metal teeth create a rotation of the cog wheel cylinder 29, when the teeth engage with the teeth of the cog wheel and provides a rotational force when the metal plate is moved forward, FIG. 7.

The metal plate is moved forth and back via a metal pipe 44, that is present along the shaft 22 and the handle 23, which metal pipe surrounds the metallic thread feeding pipe 23. In this way the shaft 22 has the possibility to rotate 360 degrees in relation to the handle. The metal plate 42 and the long metal pipe 44 are moved with the help of a device 45 that is mounted on the handle 23. The proximal end 46 of the needle 25 has four sides, suitably with quadratic cross-section and it may be mounted and placed in the four-sided channel along the longitudinal axis 32 of the cog wheel cylinder. In this way the needle will be replaceable.

### The Instrument for Facilitating a Suture Method

The suture method according to the present invention demands further instruments apart from the main suture instrument in order for it to be effective. Two further instruments facilitate and speed up the suture method. More details regarding the use of these instruments are explained under the principles of the invention. The construction and design of these instruments are described below. Observe that other constructions that apply the same principles but in another way also may be used and be feasible solutions for constructing similar laparoscopic instruments.

### 1. A Laparoscopic Double Forceps

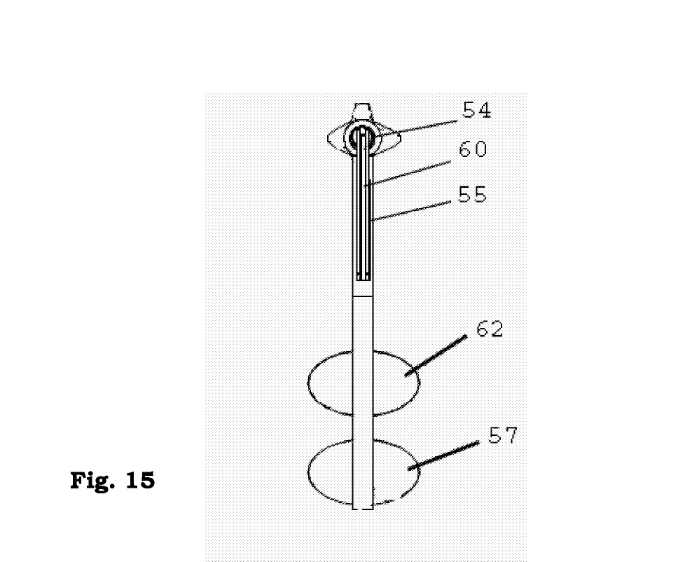

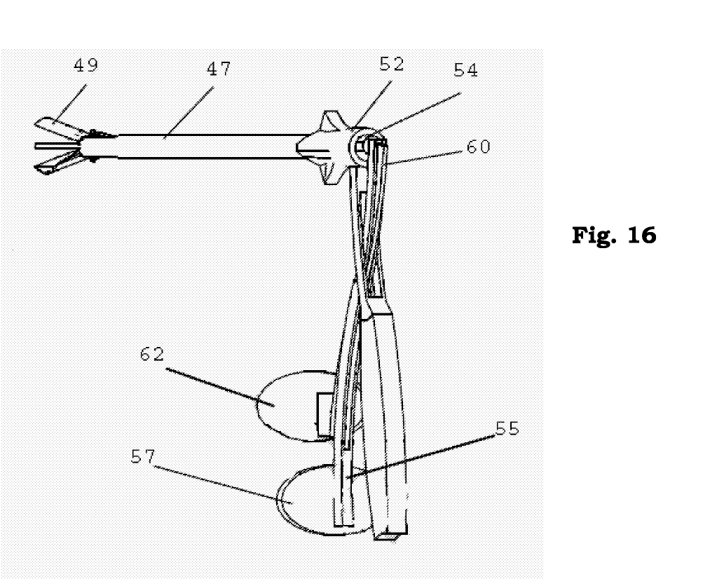

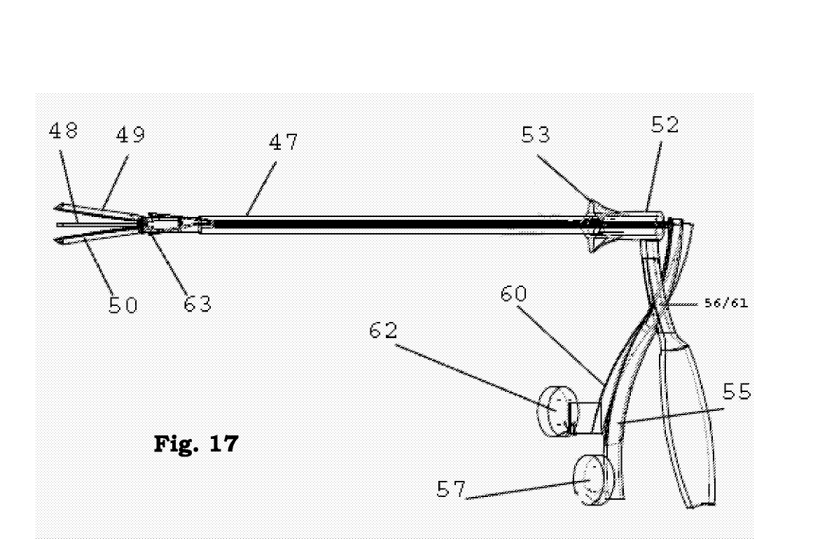

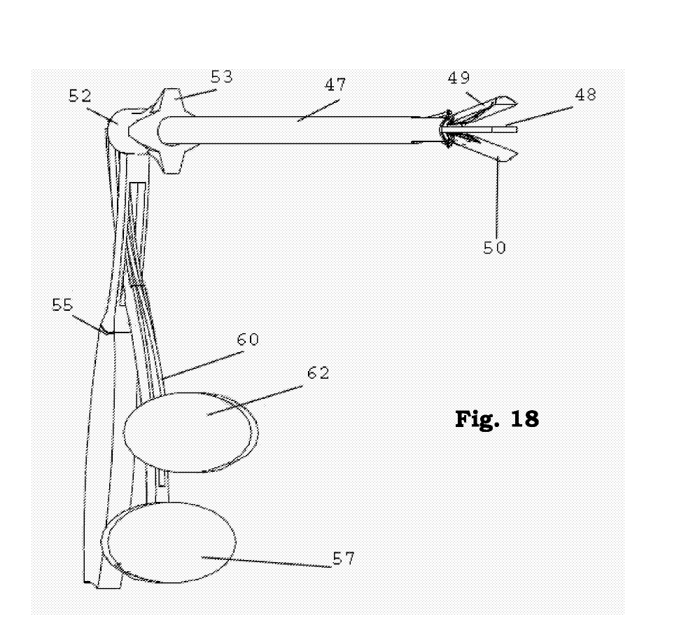

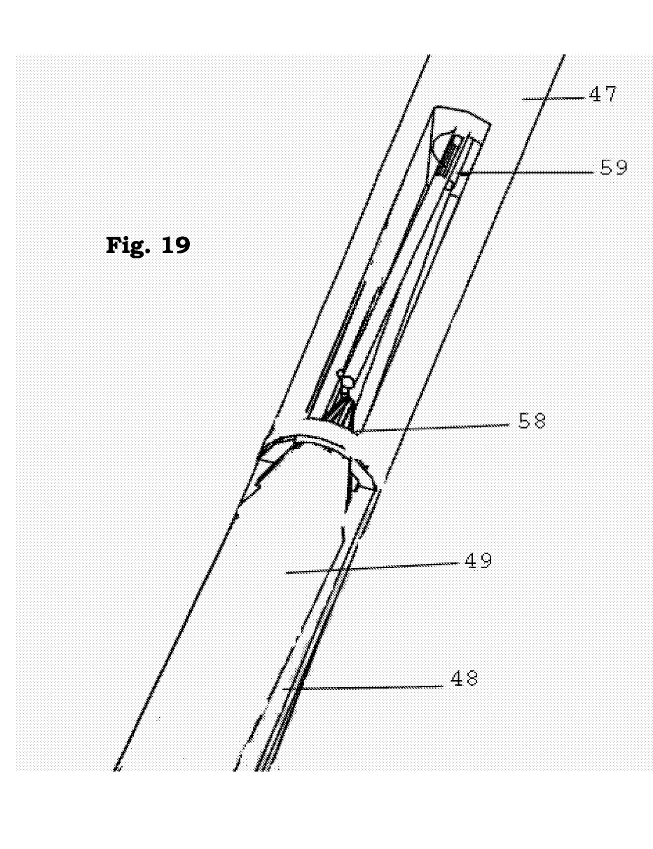

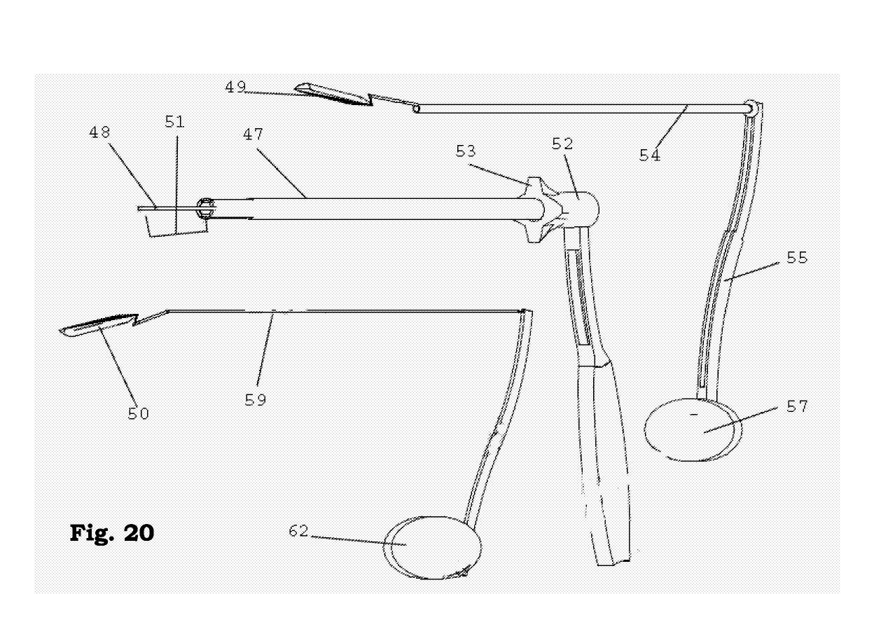

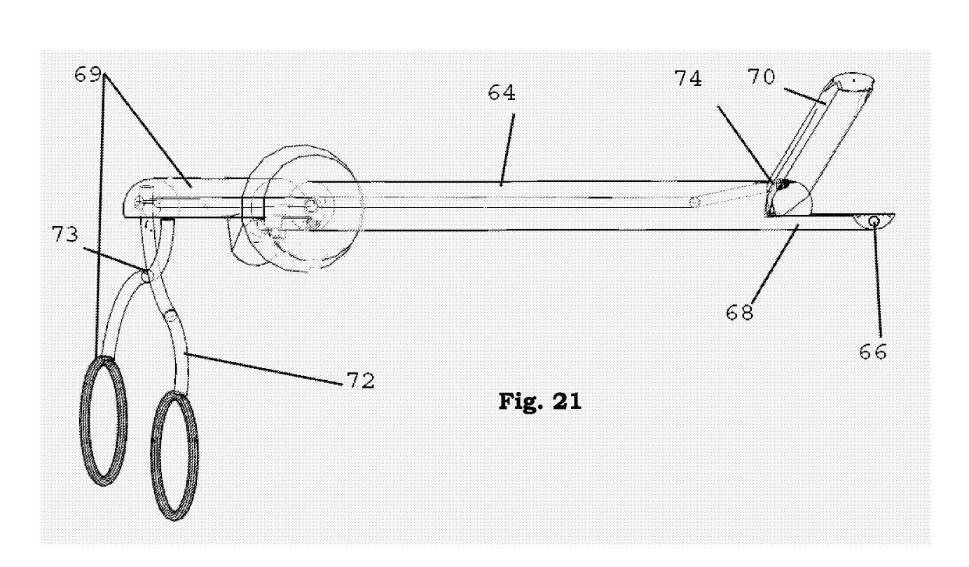

This double forceps is presented in its entirety in FIGS. 15 to 20. The double forceps consists of a chassis 47, which is a hollow metal pipe with a diameter of 5 (five) mm. the chassis of the double forceps has a thin narrow part in one end 48. This narrow part is formed as a plate. The plate is present between two jaws, an upper jaw 49 and a lower jaw 50. The plate 48 is immovable and is mounted substantially in the middle of the distal end 51 of the chassis of the double forceps. In the other end of this chassis a handle 52 is arranged, that comprises a rotation device 53, which makes the chassis together with the jaw to be rotated 360 degrees.

The upper jaw 49 is connected to a pipe 54, preferably made of metal and that has a smaller diameter and is present along the hollow part of the chassis of the double forceps and along the handle 52. The metal pipe 54 of the upper jaw is connected to a device 55, which is arranged to move around an axis 56, which is mounted in the handle 52. A lower finger button 57 controls a forward movement of this device when it is pressed by a finger. A spring that is connected to this device 55 controls a return movement of the device. Thus the upper jaw 49 is moved around an axis 58, which is mounted with the chassis of the double forceps. The upper jaw 49 is closed against the immovable metal plate 48 when the lower finger button 57 is pushed and the upper jaw is opened back to its original position with the help of a spring.

The lower jaw is connected with a metal rod 59, which is arranged along the hollow chassis of the double forceps and the handle 52 and is surrounded by the metal pipe 54, which is connected to the upper jaw. In this way the chassis of the double forceps is rotated along a rotational axis that is coupled with the handle 53. The rod 59 of the lower jaw is connected to a device 60, which moves around an axis 61, which is mounted in the handle 52. An upper finger button 62 controls a forward movement of this device when it is pushed by a finger. A spring that is connected to this device 60 controls a return movement of the device, in analogy with the movements of the upper jaw. Thus, the lower jaw 50 is moved around an axis 63, which is mounted to the chassis of the double forceps. The lower jaw 50 is closed against the immovable metal plate 48, when the finger button 62 is pushed and the lower jaw 50 is opened back to its original position with the help of a spring.

### 2. A Laparoscopic Single Forceps

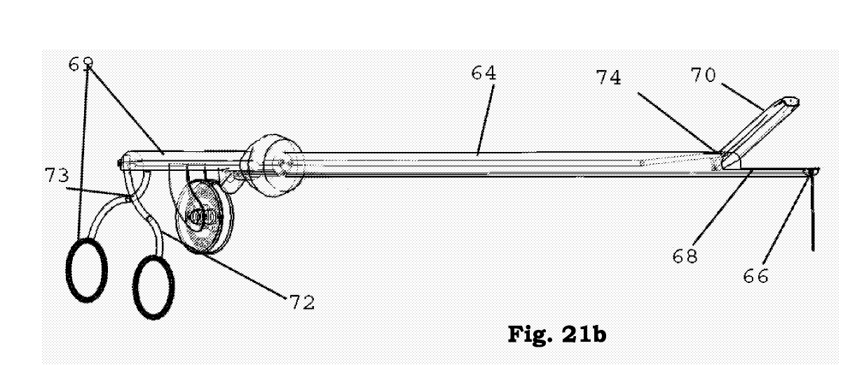

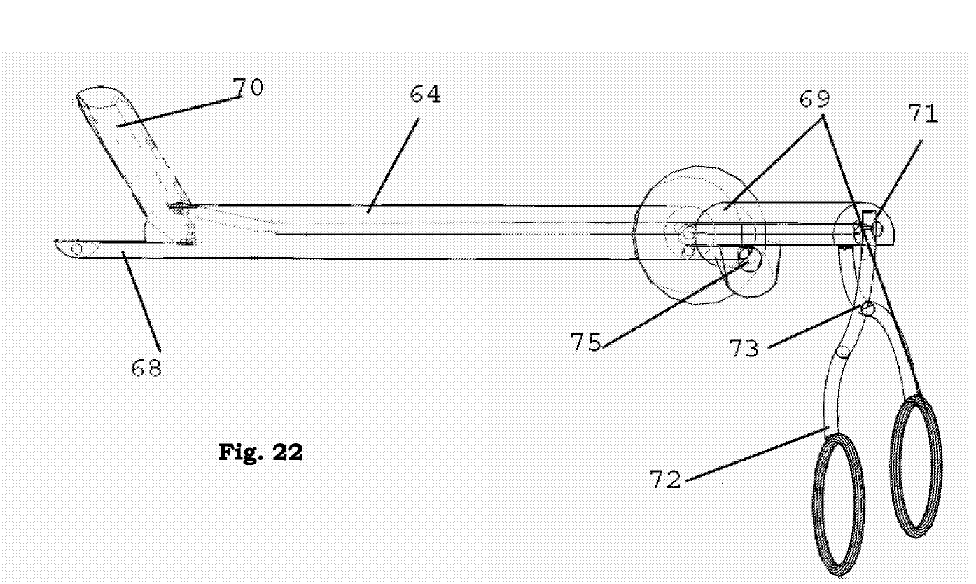

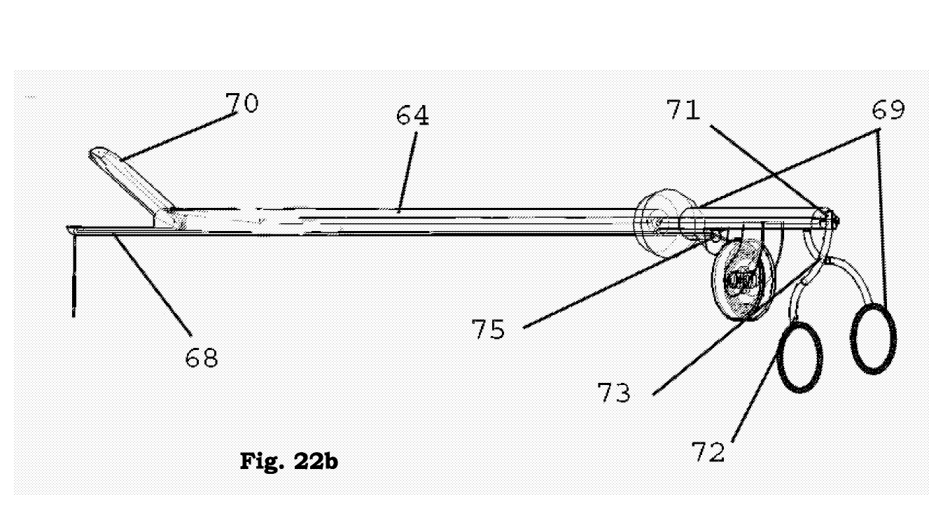

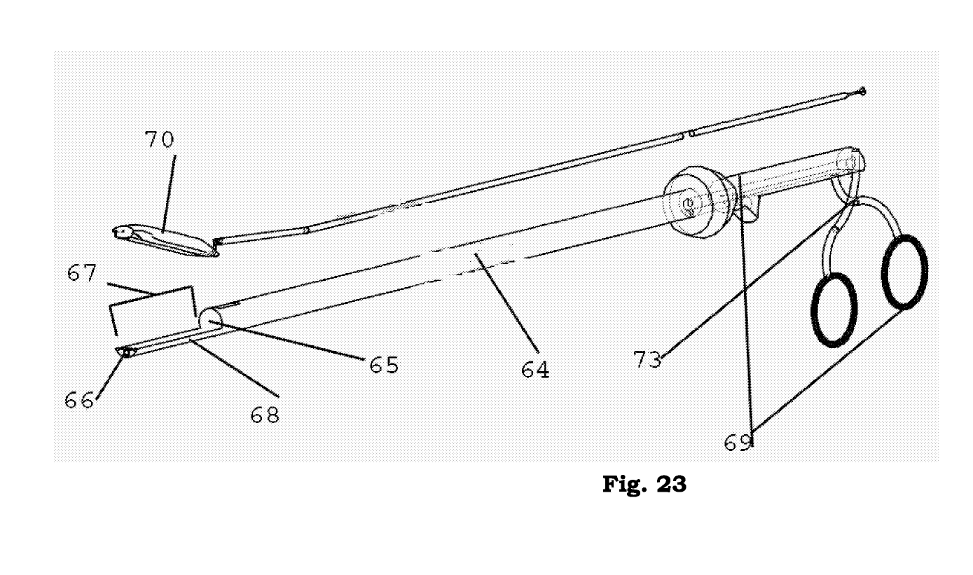

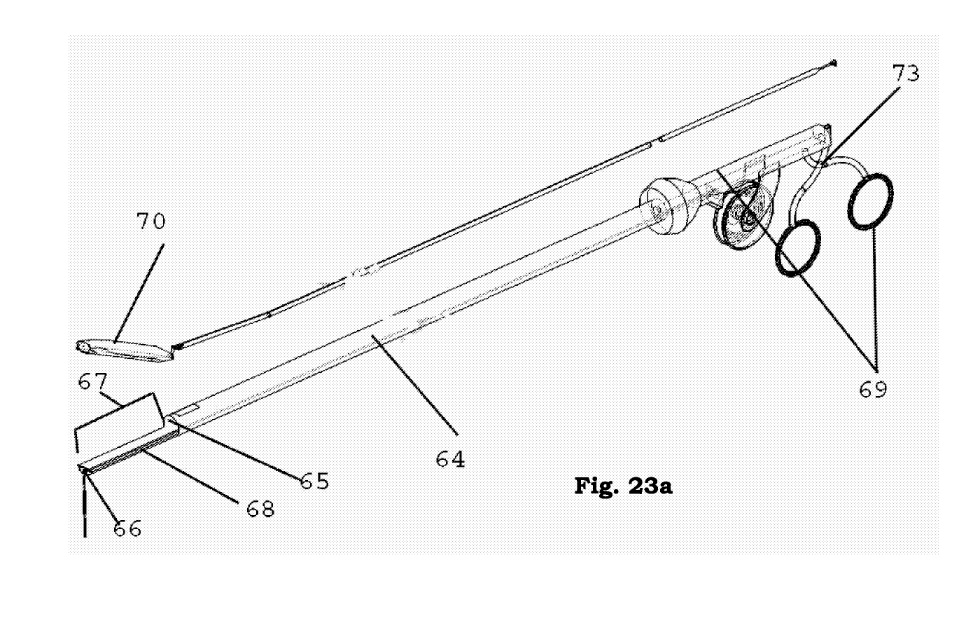

This instrument is presented in its entirety in FIGS. 21 to 23. The instrument consists of a single forceps chassis 64, which is about 5 mm in diameter and which has two channels along the whole extension of the chassis. A centre channel 65, which is arranged in the middle of the cross-section of the chassis and a thread channel 66, which is present below the centre channel (in the shown figures). A thread can be fed through the thread channel 66. The distal end 67 of the chassis of the single forceps is formed as a half circle regarding its cross-section and is flat on its upper side, whereby this part is called the immovable lower jaw 68 of the single forceps. The thread channel extends along the whole lower jaw and opens up in the distal end. The proximal end of the chassis of the single forceps is arranged with a handle 69.

Further an upper jaw 70 is arranged, which is connected to a metal rod 71, which is present along the centre channel in the chassis of the single pliers and along the handle 69. The rod 71 of the upper jaw is arranged to a jaw-controlling device 72, which is movable around an axis 73, which in turn is mounted on the handle. This device controls the movement of the jaw. The upper jaw moves around an axis 74, which is arranged to the chassis of the single forceps. The upper jaw 70 is closed against the lower immovable jaw 68, when the jaw-controlling device 72 is pulled against the handle 69. There is a tread feeding opening 75 in connection to the lower surface of the handle. This opening 75 leads to the thread channel 66.

Clips Apparatus

For a long time certain body-friendly metals have been used, such as titanium and alloys of (other metals), in connection with different operations that are performed on the human body. Different prosthesis for body parts have also been manufactured. A prosthesis is an artificial replacement for a lost or non-functional part of the human body, for example tooth prosthesis, eye prosthesis, arm or leg prosthesis. Nowadays metals in the form of clips are used for stopping bleeding and to alloy different tubular shaped tissues in the body, such as for example blood vessels and bile ducts in order to make certain operations more simple and faster. These different prosthesis and clips are nowadays manufactured of titanium, which is a body-friendly, light metal with high strength.

Up to now metal clips have been used for stopping bleeding and/or to alloy tubular shaped tissues in the body. Clips that are used today have straight arms (FIG. 24) that are pressed together with and apparatus/tool for pressing together the tubular shaped tissues.

The different clips apparatuses that up to now have been developed and are available on the market have straight jaws that press metal clips so that the arms become parallel after the pressing FIG. 25.

The present invention intends to, as stated above, to develop a new method (FIGS. 40 and 41) as well as a development of new clips and a new clips apparatus as shown in FIG. 26 and details presented in FIGS. 28 to 33. The new method intends to use a newly developed clip for alloying common operation threads/sutures and to hold their ends together and in place. For this purpose the clips are designed such that their arms cross each other as is shown in FIGS. 40 and 41.

The new clips apparatus is designed such that the jaws of the apparatus, that hold the clips, cross each other when the apparatus is in an active position in order to be able to squeeze the thread ends and to cross the arms of the clips such as in FIG. 28. The jaws have thus grooves that lead the clips such that they overlap during the squeezing. The clips can also comprise more than two arms, i.e. three or more that are placed in different planes and that overlap each other to obtain maximum locking of a suture.

The clip apparatus according to the present invention consists of two jaws 1, whereby each jaw displays a generally half circle form, and whereby the inside of each jaw 2 that holds the clips, is curved (arc-shaped) and that they are present on two different levels in relation to each other, FIG. 28. In this manner they can cross each other when they are pressed together, FIG. 28.

On the inside of the two jaws there is a groove 3 that guide the clips to their position before use. The two jaws are connected to the main body part of the apparatus, that here is called the body 4 of the apparatus. The contact between the two jaws and the body is such that it permits that the jaws are displaced backwards and forward with the aid of a long metal pipe 6. The other end of the metal pipe is connected to a handle 7, which is movable around a rotational point. This movement leads to that the jaws are displaced backwards and forward in a groove in a detachable part 8 of the body 4.

In the distal end of the body 4 are two elevated four-sided blocks 9, the aim of which is to press together the two jaws 1, when the jaws 1 are pulled back with the help of the handle 7 and the pipe 6. Further there are two slightly elevated curved metal blocks 10 between said four-sided block 9, the aim of which is to force the jaws to move apart, i.e. that they move from each other to a passive position when the jaws are pushed forward with the help of a spring device 11, that affect the handle 7 in a return direction.

In connection to the lower part of the jaws 1 are two small metal cylinders 12. Each of the cylinders is connected to a jaw from below 13. The function of the cylinders 12 in this design is to hold the jaws apart when the jaws are pushed forward so that the cylinders 12 are hooked into the slightly elevated curved metal blocks 10 that are integrated in the distal part of the body 4. In this way the jaws are rotated outwards which leads to that the jaws move apart. Further a clips container 14 containing several clips is arranged on the underside of the distal part of the body 4.

A spring 15 is arranged in the clips container 14, which spring 15 pushes one clip at the time. The spring device in the clips container pushes each clip through a four-sided hole 17 in the front part of the body. Further, a clips feeding rod 18 is arranged, which moves forward and rearward through the hole 17 in the body 4 for pressing the clips between the two jaws 1. The clips feeding rod 18 is connected to a longer rod 19, that is mounted through the long metal pipe 6 and is connected to a second handle 20, the movement of which makes the clips feeding device to move easily over the clips hole 17 in the body 4 and in this way feeds a clip one at the time in the grooves 3 arranged in the clips jaws 1.

The apparatus has an important advantage in comparison with other apparatuses in that it may be rotated 360 degrees with the help of a turning device 21 which is mounted on the handle.

### THE PRINCIPLES OF THE INVENTION

In this invention two main elements or principles are applied for sewing faster and at the same time more effective with laparoscopic instruments. These elements and methods are presented below.

### First Element in the Principles of the Invention

As first element, the needle is used for making holes and to feed the thread from one side to the other in a tissue or tissues without the needle itself having to change side. Two different methods in this invention are suggested for this.

### Second Element in the Principles of the Invention

The second element in the invention is to put the ends of the thread together without performing a traditional knot. In this case use of a metallic clip is suggested that is first rolled around the threads and then is pushed together in order to hold them fixed, see FIG. 36.

The first element comprising two methods
1. A first method: in this method, after the needle has gone through the tissues from side A to side B, a channel is created for the thread to be fed through in that the needle is hollow. After enough thread has been fed from one side A to the other side B the needle is drawn back to side A, i.e. the needle will be on the same side of the tissue as it originally was. FIG. 34 a-f shows the different steps in this process.
2. A second method: in this method the needle has a grip device for holding the thread and after the needle has gone through the tissues from side A to side B, the needle holds the thread via its grip device and feeds the thread from side B to side A. The grip device may be as is shown in FIG. 35 only a sling. Such a solution will however not be of interest for a laparoscopic operation since the pulling through of the thread will require a great precision of movements, which is not practically feasible.

An active grip device that can be opened and closed as a laparoscopic instrument is of more interest.

The two above mentioned new ways of using the needle for a laparoscopic operation are characterized in that
1. The needle will, during the whole process, be placed on the same side of the tissue and can therefore be fixedly connected to the handle.
2. In the first embodiment the needle is hollow and constitutes a channel for feeding the thread from one side of the tissue to the other.
3. In the second embodiment the needle is equipped with a grip device that preferably can be controlled, i.e. opened and closed, from outside.

In the case with needle used as a channel, which is shown in FIG. 34, it has been assumed that the thread initially is on the same side as the needle, while in the case with a needle with grip device as shown in FIG. 35, the initial position of the thread is on the opposite side of the needle. This will be shown more closely below. These two figures show that also other configurations may be possible.

As mentioned earlier, the principles outlined in FIGS. 34, 35 and 36 constitute the main feature of the presented invention. In the following text, the complete methods for facilitating suturing tissues during a laparoscopic operation are presented.

Suturing Method when a Thread is Fed Through the Needle

In this section the inventive method of laparoscopic sewing with the help of a hollow needle that is connected to a handle as in a narrow laparoscopic instrument is described. The needle constitutes a channel for feeding the thread through the tissues. It has been shown that it is preferable to have the thread feeder on the same side as the needle. The process is done as shown in FIG. 37.

As shown in FIG. 37, the tissues will hang on the thread after the four first steps. During this position the thread shall be cut and then its ends shall be brought together. In order for the continuing work to be performed in a comfortable and efficient way the use of a double forceps is suggested, as shown in FIG. 38.

Each jaw of the double pliers shown in FIG. 38 can be maneuvered separately. The immovable side is in the middle. In FIG. 39 it is shown how the continuing process may be done when a double forceps is used.

After all steps shown in FIG. 39 have been performed, the ends of the thread can be brought together. This can be done in several ways. FIG. 41 shows a new method for performing this in a fast way. Here a metallic band (metallic clip) is used, which is moved around the thread and is pressed together during simultaneous overlapping. It should be noted that the metallic clip has to be brought together and its ends have to pass/cross each other so that the threads are held in a stable way, as for example is shown in FIG. 40.

To use a metallic band/clips as shown in FIGS. 40 and 41 is a method that facilitates the process.

If so desired a laparoscopic instrument may be brought in, which holds the needle and instead bring in an ordinary laparoscopic dissecting forceps with slightly bent jaws for knotting in the conventional way, however with the help of the double forceps of the invention. The knotting can be done in different ways, whereby one is presented in FIG. 42.

Suturing by Using a Needle with Grip Device

In this section a method of sewing together tissues with the help of a needle with grip device is presented. The grip device presented here consists of a steel wire that is placed in the hollow needle and may be fed in and out, see FIG. 43. If the thread is completely inside, as in FIG. 43.b, the needle is in sewing position. If the thread is pushed out only a small part, the needle is in secured position, FIG. 43.a. The steel wire can be brought out a larger part for gripping a thread, see FIG. 43.c.

The sewing procedure with the help of a needle provided with a steel wire is shown in FIG. 44. After all steps shown in FIG. 44 have been performed, one is ready for bringing the ends of the thread together. It has been shown that a single forceps with an integrated thread feeder, such as in FIG. 45, can substantially facilitate the process shown in FIG. 44.

FIG. 46 shows the procedure during use of the instrument shown in FIG. 45.

When all steps shown in FIG. 46 have been performed the ends of the thread may be brought together with the help of a metallic band/clips as in the former case. This is shown in FIG. 47.

If so desired, such as with the former method, the thread may be knotted with the help of a single forceps. This is shown in FIG. 48. The only difference is that the thread that is wound around the laparoscopic dissecting forceps has to be the one coming from the thread feeder.

As mentioned earlier, the needle with the grip device can also be used as a forceps. In this case it may be put in secure position and work with it in exactly the same way as the forceps shown in FIG. 47. This method is schematically shown in FIG. 49.

### SUMMARY OF THE INVENTION

The invention that provides the possibility of an effective and fast laparoscopic/endoscopic suturing method consists of the following main parts:
1. A specially made laparoscopic instrument that in one end has a specially made needle through which a thread can be fed.
2. A specially made laparoscopic instrument that in one end has a specially made needle that has the capability of receiving and holding the end of the thread.
3. A special clips machine and a new clip.
4. A thread feeder, eventually integrated in a forceps.
5. A specially made double forceps.

The suture method with the help of the different instruments of the invention.

1. With a Specially Made Laparoscopic Instrument that in One End has a Specially Made Needle Through which a Thread can be Fed:

The needle is thereby simply a hollow needle. The suture method starts by feeding the needle through the tissues and then feeding the thread from one side of the tissues to the other. For a laparoscopic operation the needle has to be held with a handle. Several different designs are possible.

A special handle is arranged on which the needle is mounted before entry through the operation port. The needle has two different positions, one passive and one active. In the passive position the needle is parked in the handle and in the active position, it will have a well defined angle with the handle. It is also conceivable to have a protection that prevents the needle from hitting other tissue before the protection is removed. The same functionality can be obtained with many different mechanical solutions.

The thread needs to be fed through the needle. A special device for this must be present, such as a rotatable (W)reel that contains suture thread and that is maneuvered from the handle for feeding. The thread feeding system can also preferably contain a knife for cutting the thread.

A more simple designed suturing aid is that the needle is equipped with a deformable pipe on the backside. The needle is entered into the operation port and is then held with a laparoscopic needle handle. In contrast to the traditional method with a needle that is connected to a thread, the needle does not have to be released but the needle is held by the same needle holder during the whole suturing procedure.

2. With a Needle with Grip Device

Another alternative could be to combine a function with the needle in order to hold the end of the thread.

The grip device can consist of two or more steel wires that can be fed through the needle and the instrument. One end of the steel wire is thereby ring-shaped or forms a loop during movement through the needle when the steel wire is pushed outside the needle and the form of this loop or ring-shape is adjusted during pulling into the needle. The steel wire can also be comprised by several threads that are arranged around each other.

The problem may be solved in many different ways. One way may be to use hollow needles through which a steel wire is fed, where one end is fixedly connected on the inside of the tip of the needle. When the steel wire is fed in some centimetres, a circle of steel wire is formed. When the circle is out of the needle, the thread may be inserted through it and then pull back the steel wire. The circle becomes smaller and can thereby hold the thread.

It should be noted that this is not the only way, even if it is the most practical. It is also possible to design a needle that can separate and function as a forceps.

As above, the needle needs a handle. The needle can be separate or fixedly mounted on the handle. It could have protective means or several different mechanical methods for moving the parts from outside.

In the embodiment with a ring-shaped steel wire it may be used as protection if it is out of the tip of the needle only partly. The steel wire prevents the tip of the needle from reaching the tissues and becomes therefore a secured needle.

3. With a Special Clip

In this invention a new method is suggested for speeding up the suturing. After the thread has been fed through the tissues a metallic band is pressed around the threads and hold them together. For this a special clip is used.

4. With a Thread Feeding System

The aim with the thread feeding system is to feed thread, hold the thread and/or cut it. There can be many different mechanical solutions for the system and how to manipulate it remotely.

For the case with the grip device it has been shown that it is preferable if the thread feeder can be integrated with a forceps. In this case the process becomes faster and easier.

5. With a Double Forceps

Another instrument that substantially facilitates the work is a double forceps where each jaw can be maneuvered separately. How this can be done is a detail that will not have a unique solution.

1. Instrument to be used in laparoscopic operation, comprising a suture instrument, which suture instrument comprises a needle, characterised in that the instrument is arranged with a handle that receives and manoeuvres said needle and that the needle is arranged to let a thread pass in it, whereby the needle can be used for feeding the thread through the tissues without the needle as a whole, needs to be fed from one side of the tissue to the other.

2. Instrument according to claim 1, where the needle is arranged with a flexible, non-elastic pipe through which a thread can be fed there-through from the handle.

3. Instrument according to claim 1, where the needle is exchangeably fixed to the handle.

4. Instrument according to claim 3, where the position of the needle is arranged to be changed from a rest position to an active position via said handle.

5. Instrument according to claim 3, where the tip of the needle is arranged with a removable protective device for protection during insertion through an operation port.

6. Instrument according to claim 1, where the needle is hollow.

7. Instrument according to claim 6, whereby the needle is arranged with at least one steel wire movable in the hollow space.

8. Instrument according to claim 6, whereby the needle is arranged with a suture thread movable in the hollow space.

9. Instrument according to claim 7, where the end of the steel wire is ring-shaped, whereby the ring widens and becomes larger when the steel wire is fed out of the needle and becomes smaller and can hold a thread when it is pulled back.

10. Instrument according to claim 1, where the device for holding the thread is a hole in the needle.

11. Instrument according to claim 7, whereby the needle is arranged with a hole on its side adjacent the tip of the needle, through which hole the ring-shaped end of the steel wire or a suture is arranged to be fed out.

12. Instrument according to claim 7, whereby the needle is arranged with a hole in its tip, through which hole a ring-shaped end of a steel wire or a suture thread is arranged to be fed out.

13. Instrument according to claim 1, whereby the steel wire is present through the whole instrument and can be fed out and in via a device connected to the instrument.

14. Instrument according to claim 1, wherein it comprises a thread feeding device.

15. Instrument according to claim 14, where the thread feeding device is arranged to also break the feeding.

16. Instrument according to claim 1, wherein it further comprises a laparoscopic double forceps for use during laparoscopic operation, characterised in that each jaw is arranged to be maneuvered separately via an externally arranged guide means.

17. Double forceps according to claim 16, which consists of two movable jaws with a fixed jaw arranged between them.

18. Double forceps according to claim 16, whereby the guide means are arranged on an outer handle and are arranged to guide the two movable jaws separately.

19. Instrument according to claim 1, wherein it further comprises a laparoscopic single forceps for laparoscopic suturing comprising a device for feeding suture thread.

20. Single forceps according to claim 19, where the thread feeding is arranged to be slowed down.

21. Single forceps according to claim 19, where the device has a knife for cutting the thread.

22. Single forceps according to claim 19, having a movable jaw and a non-movable jaw for holding an end of a thread.

23. Single forceps according to claim 22, which is arranged to control the movement of the movable jaw of the single forceps.

24. Single forceps according to claim 19, including a channel that runs generally along the whole length of the instrument.

25. Single forceps according to claim 24, wherein a thread is arranged to be fed from one end to the other end.

26. Instrument according to claim 1, wherein it further comprises a laparoscopic clip instrument, characterised in that it comprises two jaws that are bent in axial direction and cross or pass each other in active position when the jaws are brought together towards each other.

27. Laparoscopic clip instrument according to claim 26, that contains a device for pressing the jaws of the instrument towards each other.

28. Laparoscopic clip instrument according to claim 26, that contains a device for pressing the jaws of the instrument towards each other when the jaws of the instrument are pulled back.

29. Laparoscopic clip instrument according to claim 26, whereby the jaws are arranged to be pushed apart when they are pushed forward.

30. Laparoscopic clip instrument according to claim 26, that displays a device for pushing back the jaws of the instrument to an initial position/passive position.

31. Laparoscopic clip instrument according to claim 26, which comprises a device for feeding clips into the bent jaw.

32. Laparoscopic clip instrument according to claim 31, which comprises a rod connected to a movable handle mounted on one end of the instrument.

33. Laparoscopic clip instrument according to claim 26, comprising a device for keeping and storing of clips when the instrument is used.

34. Laparoscopic clip instrument according to claim 33, which comprises a container magazine for clip arranged with a spring arranged to press up the clips one and one through an opening in the shaft of the clip instrument.

35. Laparoscopic clip instrument according to claim 26, whereby it contains clips with at least two arms that are in two or more levels of planes.
